(19) United States
(12) Patent Application Publication
(10) Pub. No.: US 2017/0290806 A1
(43) Pub. Date: Oct. 12, 2017

(54) PCSK9 Inhibitors and Methods of Use Thereof

(71) Applicant: Temple University-Of The Commonwealth System of Higher Education, Philadelphia, PA (US)

(72) Inventors: Magid Abou-Gharbia, Exton, PA (US); Wayne E. Childers, New Hope, PA (US)

(21) Appl. No.: 15/509,627

(22) PCT Filed: Sep. 8, 2015
(86) PCT No.: PCT/US15/48912

§ 371 Date: Mar. 8, 2017

Related U.S. Application Data
(60) Provisional application No. 62/047,456, filed on Sep. 8, 2014.
(60) Provisional application No. 62/047,462, filed on Sep. 8, 2014.

(51) Int. Cl. A61K 31/415; A01N 43/56; A61K 31/455; A61K 31/5377; A61K 31/4439; C12N 15/79

(52) U.S. Cl. CPC A61K 31/415; A61K 31/4439; C12N 15/79; A61K 31/455; A61K 31/5377; A01N 43/56; A61K 2300/00; C12Q 1/686

(57) ABSTRACT

The present invention relates to compositions and methods for treating lipid disorders in a subject. In one embodiment, the compositions of the present invention can be used to inhibit protease proprotein convertase subtilisin-like kexin type 9 (PCSK9). In another embodiment, the compositions of the present invention can be used to disrupt the protein-protein interaction between PCSK9 and low-density lipoprotein receptor (LDLR).

### CROSS REFERENCE TO RELATED APPLICATIONS

This application claims priority to U.S. Patent Application Ser. No. 62/047,456, filed Sep. 8, 2014, and U.S. Patent Application Ser. No. 62/047,462, filed Sep. 8, 2014, the entire contents of which are each incorporated herein by reference.

### BACKGROUND OF THE INVENTION

Considerable success has been achieved in treating high cholesterol by lowering LDL-C levels using statins. However, only 38% of patients taking these drugs are achieving the low-density lipoprotein cholesterol goals set by the National Cholesterol Education Program (NCEP). Furthermore, patients with homozygous familial hypercholesterolemia who have markedly elevated cholesterol levels respond poorly to current drug therapy, and are at very high risk of premature cardiovascular disease. These and other patients will dramatically benefit from an aggressive treatment of hypercholesterolemia. Compounds that block the PCSK9/LDLR interaction may be effective lipid-lowering agents. PCSK9 as a therapeutic target appears to be well validated. This is strongly supported by the low plasma LDL-C levels associated with loss-of-function mutations in the PCSK9 gene, which indicate that inhibition of PCSK9 through small molecules should be effective cholesterol-lowering drugs. In addition, no safety issues associated with inhibition of PCSK9 have been identified. Knockout mice lacking PCSK9 developed normally and have no gross neurological defects. Humans heterozygous for loss-of-function mutations in PCSK9 seem to be healthy and have a normal life-span. In addition, human heterozygote with two inactivating mutations in the PCSK9 gene (Y142X and ΔR97) and no circulating PCSK9 have very low level of LDL-C (14-34 mg dL-1) and normal hepatic and renal function. Monoclonal antibodies (mAb) have been identified as a strategy for lowering LDL-C. However, no small molecular antagonist against PCSK9 has been identified.

Thus, there is a need in the art for small molecule compounds that lower cholesterol through the inhibition of PCSK9, or by disrupting the protein-protein interaction between PCSK9 and LDLR. The present invention addresses this need in the art.

### SUMMARY OF THE INVENTION

The present invention relates to a composition comprising a compound of Formula I:

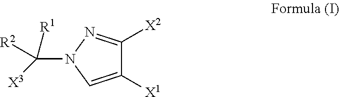

wherein in Formula (I):

R¹ is selected from the group consisting of hydrogen, C₁₋₆ alkyl, C₁₋₆ alkenyl, C₁₋₆ alkynyl, C₁₋₆ haloalkyl, optionally substituted aryl, and optionally substituted alkylaryl;

R² is selected from the group consisting of hydrogen, C₁₋₆ alkyl, C₁₋₆ alkenyl, C₁₋₆ alkynyl, C₁₋₆ haloalkyl, optionally substituted aryl, and optionally substituted alkylaryl, or R¹ and R² are optionally taken together with the atoms to which they are bound to form a ring containing 3 to 7 atoms, optionally containing a nitrogen, oxygen, or sulfur atom;

X¹ is selected from the group consisting of NR³COR⁴ and NR³SO₂R⁴

X² is selected from the group consisting of hydrogen and C₁₋₆ alkyl, or X¹ and X² are optionally taken together to form an optionally substituted aromatic six membered ring optionally containing a nitrogen atom;

X³ is selected from the group consisting of CONR³R⁴ and SO₂NR³R⁴;

R³ at each occurrence is independently selected from the group consisting of hydrogen and C₁₋₆ alkyl;

R⁴ at each occurrence is independently selected from the group consisting of hydrogen, C₁₋₆ alkyl, C₁₋₆ alkenyl, C₁₋₆ alkynyl, optionally substituted aryl, optionally substituted arylalkyl, optionally substituted heteroaryl, and optionally substituted heteroarylalkyl, or R³ and R⁴ are optionally taken together with the atoms to which they are bound to form a ring containing 3 to 7 atoms.

In one embodiment, R¹ and R² are methyl groups. In another embodiment, X¹ is NHCOR⁴, and R⁴ is selected from the group consisting of optionally substituted aryl, optionally substituted arylalkyl, optionally substituted heteroaryl, and optionally substituted heteroarylalkyl. In another embodiment, X³ is CONHCH₂R⁵, and R⁵ is selected from the group consisting of optionally substituted aryl, optionally substituted arylalkyl, optionally substituted heteroaryl, and optionally substituted heteroarylalkyl. In another embodiment, the compound of Formula (I) is a compound of Formula (II) or Formula (III):

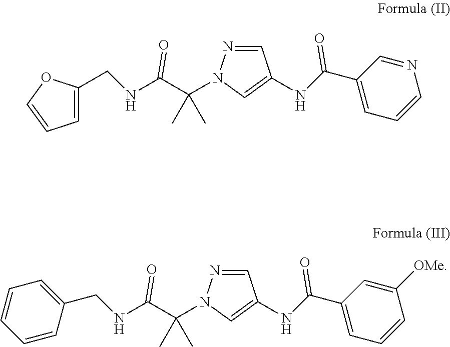 [SMILES: CC(C)(C(=O)NCc1ccco1)n1cc(NC(=O)c2cccnc2)cn1 ; COc1cccc(C(=O)Nc2cnn(C(C)(C)C(=O)NCc3ccccc3)c2)c1]

The present invention also relates to a composition comprising a compound of Formula (IV):

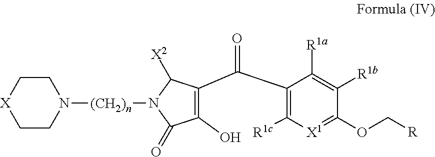

wherein in Formula (IV),

n is an integer from 1 to 6;

R is selected from the group consisting of optionally substituted aryl and optionally substituted heteroaryl;

R^{1a}, R^{1b}, R^{1c}, and R^{1d} are at each occurrence independently selected from the group consisting of hydrogen, halogen, optionally substituted C₁₋₆ alkyl, optionally substituted C₁₋₆ alkenyl, optionally substituted C₁₋₆ alkynyl, optionally substituted C₁₋₆ haloalkyl, optionally substituted C₃₋₇ cycloalkyl, optionally substituted C₁₋₆ alkoxy, cyano, nitro, OR², SR³, SO₂R³, NR^{4a}R^{4b}, NR^{4a}COR⁵, NR^{4a}CONR^{4a}R^{4b}, NR^{4a}COOR⁶, SO₂NR^{4a}R^{4b}, and NR^{4a}SO₂R⁶;

X is selected from the group consisting of oxygen, sulfur, NR^{4a}, NCOR⁵, NCONR^{4a}R^{4b}, NCOOR⁶, and NR^{4a}SO₂R³;

X¹ is selected from the group consisting of N and CR^{1d};

X² is selected from the group consisting of optionally substituted aryl and optionally substituted heteroaryl;

R² at each occurrence is independently selected from the group consisting of hydrogen, optionally substituted C₁₋₆ alkyl, optionally substituted C₁₋₆ alkenyl, optionally substituted C₁₋₆ alkynyl, optionally substituted C₁₋₆ haloalkyl, optionally substituted C₃₋₇ cycloalkyl, optionally substituted aryl, COR⁵, CONR^{4a}R^{4b}, SO₂NH₂, and SO₂R³;

R³ at each occurrence is independently selected from the group consisting of hydrogen, optionally substituted C₁₋₆ alkyl, optionally substituted C₁₋₆ alkenyl, optionally substituted C₁₋₆ alkynyl, optionally substituted C₁₋₆ haloalkyl, optionally substituted C₃₋₇ cycloalkyl, and optionally substituted aryl;

R^{4a} and R^{4b} at each occurrence are independently selected from the group consisting of hydrogen, optionally substituted C₁₋₆ alkyl, optionally substituted C₁₋₆ alkenyl, optionally substituted C₁₋₆ alkynyl, optionally substituted C₁₋₆ haloalkyl, optionally substituted C₃₋₇ cycloalkyl, and optionally substituted aryl;

R⁵ at each occurrence is independently selected from the group consisting of hydrogen, optionally substituted C₁₋₆ alkyl, optionally substituted C₁₋₆ alkenyl, optionally substituted C₁₋₆ alkynyl, optionally substituted C₁₋₆ haloalkyl, optionally substituted C₃₋₇ cycloalkyl, and optionally substituted aryl; and

R⁶ at each occurrence is independently selected from the group consisting of optionally substituted C₁₋₆ alkyl, optionally substituted C₁₋₆ alkenyl, optionally substituted C₁₋₆ alkynyl, optionally substituted C₁₋₆ haloalkyl, optionally substituted C₃₋₇ cycloalkyl, and optionally substituted aryl.

In one embodiment, n is 3 and X is oxygen. In another embodiment, X² is 4-pyridyl. In another embodiment, R is phenyl. In another embodiment, the compound of Formula (IV) is a compound of Formula (V):

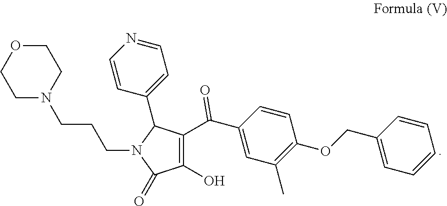 [SMILES: Cc1cc(C(=O)C2=C(O)C(=O)N(CCCN3CCOCC3)C2c2ccncc2)ccc1OCc1ccccc1]

The present invention also relates to a method for inhibiting proprotein convertase subtilisin-like kexin type 9 (PCSK9) in a subject. The method includes administering a therapeutically effective amount of a composition of the invention.

The present invention also relates to a method for disrupting the protein-protein interaction between PCSK9 and low-density lipoprotein receptor (LDLR). The method includes administering a therapeutically effective amount of a composition of the invention.

The present invention also relates to a method for treating a lipid disorder in a subject. The method includes administering a therapeutically effective amount of a composition of the invention to a subject. In one embodiment, the lipid disorder is hypercholesterolemia. In one embodiment, the composition further comprises an additional therapeutic agent selected from the group consisting of an HMGCoA reductase inhibitor, a nicotinic acid, a fibric acid, and a bile acid-binding resin.

### BRIEF DESCRIPTION OF THE DRAWINGS

The following detailed description of preferred embodiments of the invention will be better understood when read in conjunction with the appended drawings. For the purpose of illustrating the invention, there are shown in the drawings embodiments which are presently preferred. It should be understood, however, that the invention is not limited to the precise arrangements and instrumentalities of the embodiments shown in the drawings.

FIG. 1 is an image of the positions of the naturally occurring PCSK9 mutations associated with elevated (top) or reduced (bottom) LDL-C levels.

FIG. 2 is an image of the overall structure of PCSK9.

FIG. 3 is an image of the prodomain inhibitory peptide binding in the catalytic site.

FIG. 4, comprising FIGS. 4A-4B, depicts images of PCSK9 and the LDL receptor. FIG. 4A is an image of a model of potential PCSK9-mediated trafficking of the LDLR. The EGF-A domain of the LDLR is required for proper trafficking of the LDL receptor back to the cell membrane. The EGF-A domain may contain a sorting signal that interacts with an endosomal protein (green star), directing the LDLR back to the cell surface on recycling endosomes (green arrows). Binding of PCSK9 might interfere with that signal, preventing the LDLR from returning to the cell surface. Alternatively, PCSK9 could contain a distinct sorting signal (red star) that results in the sorting of the PCSK9-LDLR complex (red arrows) to lysosomes. FIG. 4B is an image of the structure of PCSK9 and that of the LDLR at the endosomal PH. The LDLR is folded back upon itself at low PH leaving the EGF-A domain that bind PCSK9 exposed. The affinity of the PCSK9 for the LDLR is known to increase dramatically in the acidic environment of the endosome. The contact residues of the LDLR and the PCSK9 residues are shown.

FIG. 5 is an image of PCSK9, with the prodomain (magenta), the subtilisin-like catalytic domain (green), and the C-terminal domain (brown), and the EGF-A domain of LDLR (blue) represented as a ribbon diagram. The bound calcium ion within the EGF-A domain is shown as a red sphere.

FIG. 6 is an image of the surface of PCSK9 buried upon binding to EGF-A colored according to element type: carbon, orange; nitrogen, blue; oxygen, red; sulfur, green. Areas of PCSK9 not involved in binding are colored gray. EGF-A is represented as a stick model. Residues within EGF-A involved in binding are colored according to element type: carbon, yellow; nitrogen, blue; oxygen, red. Residues not involved in binding are colored gray.

FIG. 7 is an image of experimental data demonstrating the increased degradation of the LDLR by PCSK9. HEK-293T cells were seeded in a DMEM containing 10% Fetal Bovine Serum media and incubated overnight at 37° C. Cells were transiently transfected with Mock (lanes 1 and 2), PCSK9 (lanes 3 and 4), LDLR & PCSK9 (lanes 5 and 6), LDLR (lanes 7 and 8) cDNA constructs using the Lipofectamine-LTX as described by the manufactures (Invitrogen). Cells were incubated for additional 72 hrs, and cells and media were analyzed using western blot analysis. For analysis of the media, cells were replaced with DMEM/ITS and incubated for 5 hrs. The cell media was collected and the PCSK9 secreted into the media were precipitated using TCA, analysis and quantitated by western blot. For western blots, all samples were resolved on denaturing 15% acrylamide gels with a 4% acrylamide stacking layer. Gels were transferred to Immoblin™ nitrocellulose (Millipore) using a Trans-Blot® SD semi-dry transfer cell (BioRad). After transfer, blots were blocked with 5% non-fat milk in PBS pH 7.5+0.05% Tween-20 (PBST) for one hour. Following blocking, the blots were incubated in 1:1000 antibody (Sigma) in PBST+milk for one hour. Blots were washed with PBST three times and incubated with anti-rabbit HRP conjugated antibody (Sigma) in PBST+milk for one hour. Blots were again washed three times with PBST. HRP conjugates were detected with SuperSignal® West Pico chemoluminescent substrate (Pierce). Blots were imaged and bans were quantitated using a LAS-3000 (Fujifilm Life Science). Cell viability were assayed using Resazurin (Sigma 199303) assay and read using the Envision 2101 Multi-label plate reader with Exitation, Bodipy TMR FP 531 and Emission Rohdamine 590.

FIG. 8 is a table of experimental data demonstrating the effect of different hits on LDLR upregulation in HEK293 transfected cells. HEK-293T cells were seeded into 96 well plates in a DMEM containing 10% Fetal Bovine Serum media and incubated overnight at 37° C. Cells were transiently transfected with LDLR and PCSK9 cDNA constructs using the Lipofectamine-LTX. Compounds (1-50 uM) were added, followed by additional 43 hours of incubation. The cells were lysed and analyzed for LDLR expression and cell viability determined as described in FIG. 7.

FIG. 9 is a table of experimental data demonstrating effect of various concentrations of different hits on LDLR upregulation in HEK293 transfected cells. HEK-293T cells were seeded into 96 well plates in a DMEM containing 10% Fetal Bovine Serum media and incubated overnight at 37° C. Cells were transiently transfected with LDLR and PCSK9 cDNA constructs using the Lipofectamine-LTX. Compounds (1-50 uM) were added, followed by additional 43 hours of incubation. The cells were lysed and analyzed for LDLR expression and cell viability determined as described in FIG. 8. The three compounds exhibited a concentration dependent up-regulation of LDLR with an EC50 of 3.5, 1.6, and 3.1 μM. Compound SBC-130,022 has no effect in up-regulating LDLR.

FIG. 10 is a table of experimental data demonstrating the effect of various concentrations of different hits on LDLR upregulation in HepG2 cells. HepG2 cells were seeded into 96 well plates in a MEM containing 10% Fetal Bovine Serum media and incubated overnight at 37° C. Cells were transiently transfected with PCSK9 cDNA constructs using the Lipofectamine-LTX. Compounds (1-50 uM) were added, followed by additional 43 hours of incubation. The cells were lysed and analyzed for LDLR expression and cell viability determined as described in FIG. 8. The three compounds exhibited a concentration dependent up-regulation of LDLR with an EC50 of 2.0, 2.9 and 0.43 μM.

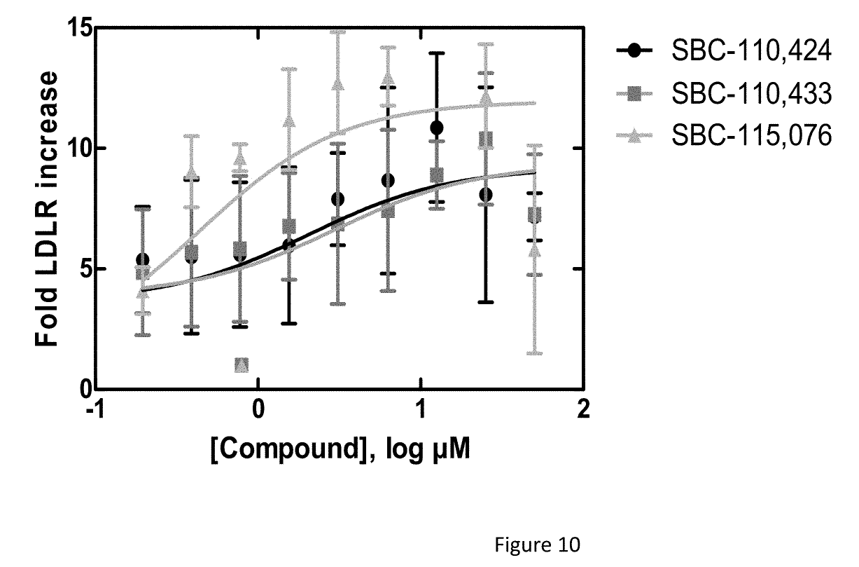

FIG. 11 is a table of experimental data demonstrating the effect of different hits on the PCSK9 processing and secretion. HEK-293T cells were seeded into 96-well plates in a DMEM containing 10% Fetal Bovine Serum media and incubated overnight at 37° C. Cells were transiently transfected with cDNA construct using the Lipofectamine-LTX. Compounds (50 uM) were added, followed by additional 43 hours of incubation. Prior to the PCSK9 assay, the cell media was replaced with the DMEM/ITS serum free media containing the same concentration of compounds or vehicle, and incubated for additional 5 hrs. The cell media was analyzed for PCSK9 secretion and cell viability determined as described above. These 4 compounds exhibited no effect on PCSK9 secretion.

FIG. 12 depicts exemplary compounds selected from compound screening.

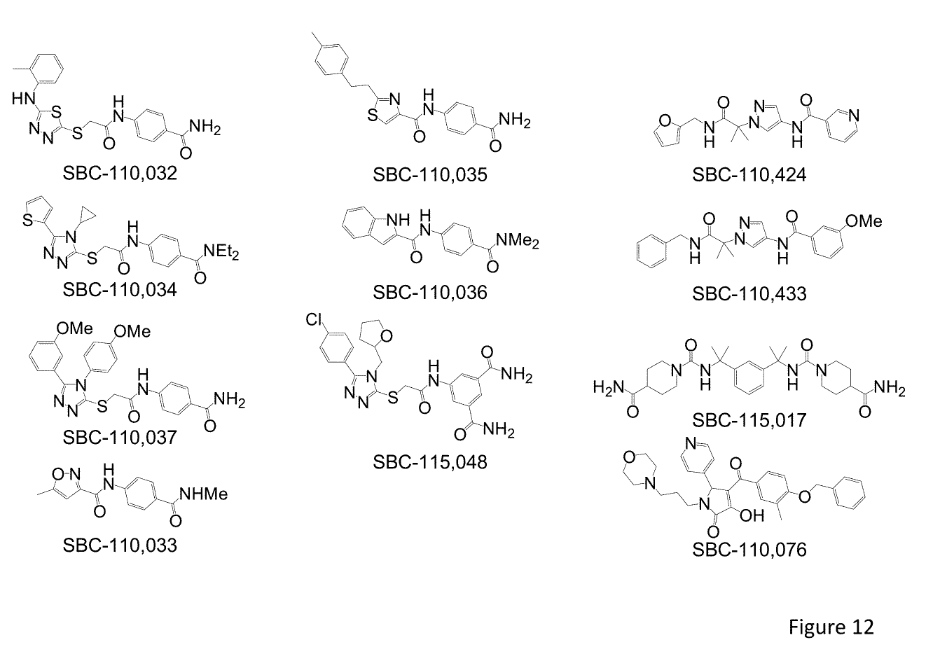

FIG. 13, comprising FIGS. 13A-13D, depicts the docking of SBC-110,424 and SBC-110,076 to PSCK9. FIG. 13A is an image of the docking of SBC-110,424 to PSCK9. FIG. 13B is an image of the docking of SBC-110,424 to PSCK9. FIG. 13C is an image of the docking of SBC-110,076 to PSCK9. FIG. 13D is an image of the docking of SBC-110,076 to PSCK9.

FIG. 14 is an exemplary sequence for the synthesis of compounds 5.

FIG. 15 is an exemplary sequence for the synthesis of compounds 5.

FIG. 16 is an image of exemplary variations to the scaffold of SBC-110,424.

FIG. 17 is an exemplary sequence for the synthesis of 2H-indazoles 13.

FIG. 18 is an exemplary sequence for the synthesis of pyrrazolopyridines 14.

FIG. 19 is an exemplary sequence for the synthesis of amino analogs of SBC-110,424.

FIG. 20 is an exemplary sequence for examining the SAR of the germinal dimethyl groups of SBC-110,424.

FIG. 21 is an exemplary sequence for the synthesis of exemplary analogs of SBC-110,076.

FIG. 22 is an exemplary sequence for the synthesis of keto-ester intermediates 31.

### DETAILED DESCRIPTION

It is to be understood that the figures and descriptions of the present invention have been simplified to illustrate elements that are relevant for a clear understanding of the present invention, while eliminating, for the purpose of clarity, many other elements found in the art related to organic chemistry, treatments for coronary artery disease, and the like. Those of ordinary skill in the art may recognize that other elements and/or steps are desirable and/or required in implementing the present invention. However, because such elements and steps are well known in the art, and because they do not facilitate a better understanding of the present invention, a discussion of such elements and steps is not provided herein. The disclosure herein is directed to all such variations and modifications to such elements and methods known to those skilled in the art.

### Definitions

Unless defined otherwise, all technical and scientific terms used herein have the same meaning as commonly understood by one of ordinary skill in the art to which this invention belongs. Although any methods, materials and components similar or equivalent to those described herein can be used in the practice or testing of the present invention, the preferred methods and materials are described.

As used herein, each of the following terms has the meaning associated with it in this section.

The articles “a” and “an” are used herein to refer to one or to more than one (i.e., to at least one) of the grammatical object of the article. By way of example, “an element” means one element or more than one element.

“About” as used herein when referring to a measurable value such as an amount, a temporal duration, and the like, is meant to encompass variations of ±20%, ±10%, ±5%, ±1%, or ±0.1% from the specified value, as such variations are appropriate.

Throughout the description, where compositions are described as having, including, or comprising specific components, or where processes are described as having, including, or comprising specific process steps, it is contemplated that compositions of the present teachings also consist essentially of, or consist of, the recited components, and that the processes of the present teachings also consist essentially of, or consist of, the recited processing steps.

In the application, where an element or component is said to be included in and/or selected from a list of recited elements or components, it should be understood that the element or component can be any one of the recited elements or components and can be selected from the group consisting of two or more of the recited elements or components.

The use of the singular herein includes the plural (and vice versa) unless specifically stated otherwise. In addition, where the use of the term “about” is before a quantitative value, the present teachings also include the specific quantitative value itself, unless specifically stated otherwise.

It should be understood that the order of steps or order for performing certain actions is immaterial so long as the present teachings remain operable. Moreover, two or more steps or actions can be conducted simultaneously.

As used herein, the term “halogen” shall mean chlorine, bromine, fluorine and iodine.

As used herein, unless otherwise noted, “alkyl” and/or “aliphatic” whether used alone or as part of a substituent group refers to straight and branched carbon chains having 1 to 20 carbon atoms or any number within this range, for example 1 to 6 carbon atoms or 1 to 4 carbon atoms. Designated numbers of carbon atoms (e.g. C₁₋₆) shall refer independently to the number of carbon atoms in an alkyl moiety or to the alkyl portion of a larger alkyl-containing substituent. Non-limiting examples of alkyl groups include methyl, ethyl, n-propyl, iso-propyl, n-butyl, sec-butyl, iso-butyl, tent-butyl, and the like. Alkyl groups can be optionally substituted. Non-limiting examples of substituted alkyl groups include hydroxymethyl, chloromethyl, trifluoromethyl, aminomethyl, 1-chloroethyl, 2-hydroxyethyl, 1,2-difluoroethyl, 3-carboxypropyl, and the like. In substituent groups with multiple alkyl groups such as (C₁₋₆alkyl)₂amino, the alkyl groups may be the same or different.

As used herein, the terms “alkenyl” and “alkynyl” groups, whether used alone or as part of a substituent group, refer to straight and branched carbon chains having 2 or more carbon atoms, preferably 2 to 20, wherein an alkenyl chain has at least one double bond in the chain and an alkynyl chain has at least one triple bond in the chain. Alkenyl and alkynyl groups can be optionally substituted. Nonlimiting examples of alkenyl groups include ethenyl, 3-propenyl, 1-propenyl (also 2-methylethenyl), isopropenyl (also 2-methylethen-2-yl), buten-4-yl, and the like. Nonlimiting examples of substituted alkenyl groups include 2-chloroethenyl (also 2-chlorovinyl), 4-hydroxybuten-1-yl, 7-hydroxy-7-methyloct-4-en-2-yl, 7-hydroxy-7-methyloct-3,5-dien-2-yl, and the like. Nonlimiting examples of alkynyl groups include ethynyl, prop-2-ynyl (also propargyl), propyn-1-yl, and 2-methyl-hex-4-yn-1-yl. Nonlimiting examples of substituted alkynyl groups include, 5-hydroxy-5-methylhex-3-ynyl, 6-hydroxy-6-methylhept-3-yn-2-yl, 5-hydroxy-5-ethylhept-3-ynyl, and the like.

As used herein, the term “cycloalkyl,” whether used alone or as part of another group, refers to a non-aromatic carbon-containing ring including cyclized alkyl, alkenyl, and alkynyl groups, e.g., having from 3 to 14 ring carbon atoms, preferably from 3 to 7 or 3 to 6 ring carbon atoms, or even 3 to 4 ring carbon atoms, and optionally containing one or more (e.g., 1, 2, or 3) double or triple bond. Cycloalkyl groups can be monocyclic (e.g., cyclohexyl) or polycyclic (e.g., containing fused, bridged, and/or spiro ring systems), wherein the carbon atoms are located inside or outside of the ring system. Any suitable ring position of the cycloalkyl group can be covalently linked to the defined chemical structure. Cycloalkyl rings can be optionally substituted. Nonlimiting examples of cycloalkyl groups include: cyclopropyl, 2-methyl-cyclopropyl, cyclopropenyl, cyclobutyl, 2,3-dihydroxycyclobutyl, cyclobutenyl, cyclopentyl, cyclopentenyl, cyclopentadienyl, cyclohexyl, cyclohexenyl, cycloheptyl, cyclooctanyl, decalinyl, 2,5-dimethylcyclopentyl, 3,5-dichlorocyclohexyl, 4-hydroxycyclohexyl, 3,3,5-trimethylcyclohex-1-yl, octahydropentalenyl, octahydro-1H-indenyl, 3a,4,5,6,7,7a-hexahydro-3H-inden-4-yl, decahydroazulenyl; bicyclo[6.2.0]decanyl, decahydronaphthalenyl, and dodecahydro-1H-fluorenyl. The term “cycloalkyl” also includes carbocyclic rings which are bicyclic hydrocarbon rings, non-limiting examples of which include, bicyclo-[2.1.1]hexanyl, bicyclo[2.2.1]heptanyl, bicyclo[3.1.1]heptanyl, 1,3-dimethyl[2.2.1]heptan-2-yl, bicyclo[2.2.2]octanyl, and bicyclo[3.3.3]undecanyl.

As used herein, the term “haloalkyl” is intended to include both branched and straight-chain saturated aliphatic hydrocarbon groups having the specified number of carbon atoms, substituted with 1 or more halogen. Haloalkyl groups include perhaloalkyl groups, wherein all hydrogens of an alkyl group have been replaced with halogens (e.g., —CF₃, —CF₂CF₃). Haloalkyl groups can optionally be substituted with one or more substituents in addition to halogen. Examples of haloalkyl groups include, but are not limited to, fluoromethyl, dichloroethyl, trifluoromethyl, trichloromethyl, pentafluoroethyl, and pentachloroethyl groups.

As used herein, the term “alkoxy” refers to the group —O-alkyl, wherein the alkyl group is as defined above. Alkoxy groups optionally may be substituted. The term C₃-C₆ cyclic alkoxy refers to a ring containing 3 to 6 carbon atoms and at least one oxygen atom (e.g., tetrahydrofuran, tetrahydro-2H-pyran). C₃-C₆ cyclic alkoxy groups optionally may be substituted.

As used herein, the term “aryl,” wherein used alone or as part of another group, is defined herein as an unsaturated, aromatic monocyclic ring of 6 carbon members or to an unsaturated, aromatic polycyclic ring of from 10 to 14 carbon members. Aryl rings can be, for example, phenyl or naphthyl ring each optionally substituted with one or more moieties capable of replacing one or more hydrogen atoms. Non-limiting examples of aryl groups include: phenyl, naphthylen-1-yl, naphthylen-2-yl, 4-fluorophenyl, 2-hydroxyphenyl, 3-methylphenyl, 2-amino-4-fluorophenyl, 2-(N,N-diethylamino)phenyl, 2-cyanophenyl, 2,6-di-tert-butylphenyl, 3-methoxyphenyl, 8-hydroxynaphthylen-2-yl 4,5-dimethoxynaphthylen-1-yl, and 6-cyano-naphthylen-1-yl. Aryl groups also include, for example, phenyl or naphthyl rings fused with one or more saturated or partially saturated carbon rings (e.g., bicyclo[4.2.0]octa-1,3,5-trienyl, indanyl), which can be substituted at one or more carbon atoms of the aromatic and/or saturated or partially saturated rings.

As used herein, the terms “arylalkyl” or “aralkyl” refers to the group -alkyl-aryl, where the alkyl and aryl groups are as defined herein. Aralkyl groups of the present invention are optionally substituted. Examples of arylalkyl groups include, for example, benzyl, 1-phenylethyl, 2-phenylethyl, 3-phenylpropyl, 2-phenylpropyl, fluorenylmethyl and the like.

As used herein, the terms “heterocyclic” and/or “heterocycle” and/or “heterocylyl,” whether used alone or as part of another group, are defined herein as one or more ring having from 3 to 20 atoms wherein at least one atom in at least one ring is a heteroatom selected from nitrogen (N), oxygen (O), or sulfur (S), and wherein further the ring that includes the heteroatom is non-aromatic. In heterocycle groups that include 2 or more fused rings, the non-heteroatom bearing ring may be aryl (e.g., indolinyl, tetrahydroquinolinyl, chromanyl). Exemplary heterocycle groups have from 3 to 14 ring atoms of which from 1 to 5 are heteroatoms independently selected from nitrogen (N), oxygen (O), or sulfur (S). One or more N or S atoms in a heterocycle group can be oxidized. Heterocycle groups can be optionally substituted.

Non-limiting examples of heterocyclic units having a single ring include: diazirinyl, aziridinyl, urazolyl, azetidinyl, pyrazolidinyl, imidazolidinyl, oxazolidinyl, isoxazolinyl, isoxazolyl, thiazolidinyl, isothiazolyl, isothiazolinyl oxathiazolidinonyl, oxazolidinonyl, hydantoinyl, tetrahydrofuranyl, pyrrolidinyl, morpholinyl, piperazinyl, piperidinyl, dihydropyranyl, tetrahydropyranyl, piperidin-2-onyl (valerolactam), 2,3,4,5-tetrahydro-1H-azepinyl, 2,3-dihydro-1H-indole, and 1,2,3,4-tetrahydro-quinoline. Non-limiting examples of heterocyclic units having 2 or more rings include: hexahydro-1H-pyrrolizinyl, 3a,4,5,6,7,7a-hexahydro-1H-benzo[d]imidazolyl, 3a,4,5,6,7,7a-hexahydro-1H-indolyl, 1,2,3,4-tetrahydroquinolinyl, chromanyl, isochromanyl, indolinyl, isoindolinyl, and decahydro-1H-cycloocta[b]pyrrolyl.

As used herein, the term “heteroaryl,” whether used alone or as part of another group, is defined herein as one or more rings having from 5 to 20 atoms wherein at least one atom in at least one ring is a heteroatom chosen from nitrogen (N), oxygen (O), or sulfur (S), and wherein further at least one of the rings that includes a heteroatom is aromatic. In heteroaryl groups that include 2 or more fused rings, the non-heteroatom bearing ring may be a carbocycle (e.g., 6,7-Dihydro-5H-cyclopentapyrimidine) or aryl (e.g., benzofuranyl, benzothiophenyl, indolyl). Exemplary heteroaryl groups have from 5 to 14 ring atoms and contain from 1 to 5 ring heteroatoms independently selected from nitrogen (N), oxygen (O), or sulfur (S). One or more N or S atoms in a heteroaryl group can be oxidized. Heteroaryl groups can be substituted. Non-limiting examples of heteroaryl rings containing a single ring include: 1,2,3,4-tetrazolyl, [1,2,3]triazolyl, [1,2,4]triazolyl, triazinyl, thiazolyl, 1H-imidazolyl, oxazolyl, furanyl, thiopheneyl, pyrimidinyl, 2-phenylpyrimidinyl, pyridinyl, 3-methylpyridinyl, and 4-dimethylaminopyridinyl. Non-limiting examples of heteroaryl rings containing 2 or more fused rings include: benzofuranyl, benzothiophenyl, benzoxazolyl, benzthiazolyl, benztriazolyl, cinnolinyl, naphthyridinyl, phenanthridinyl, 7H-purinyl, 9H-purinyl, 6-amino-9H-purinyl, 5H-pyrrolo[3,2-d]pyrimidinyl, 7H-pyrrolo[2,3-d]pyrimidinyl, pyrido[2,3-d]pyrimidinyl, 2-phenylbenzo[d]thiazolyl, 1H-indolyl, 4,5,6,7-tetrahydro-1-H-indolyl, quinoxalinyl, 5-methylquinoxalinyl, quinazolinyl, quinolinyl, 8-hydroxy-quinolinyl, and isoquinolinyl.

One non-limiting example of a heteroaryl group as described above is C₁-C₅ heteroaryl, which has 1 to 5 carbon ring atoms and at least one additional ring atom that is a heteroatom (preferably 1 to 4 additional ring atoms that are heteroatoms) independently selected from nitrogen (N), oxygen (O), or sulfur (S). Examples of C₁-C₅ heteroaryl include, but are not limited to, triazinyl, thiazol-2-yl, thiazol-4-yl, imidazol-1-yl, 1H-imidazol-2-yl, 1H-imidazol-4-yl, isoxazolin-5-yl, furan-2-yl, furan-3-yl, thiophen-2-yl, thiophen-4-yl, pyrimidin-2-yl, pyrimidin-4-yl, pyrimidin-5-yl, pyridin-2-yl, pyridin-3-yl, and pyridin-4-yl.

As used herein, the term “heteroarylalkyl” or “heteroaralkyl” refers to the group -alkyl-aryl, where the alkyl and aryl groups are as defined herein.

Unless otherwise noted, when two substituents are taken together to form a ring having a specified number of ring atoms (e.g., R2 and R3 taken together with the nitrogen (N) to which they are attached to form a ring having from 3 to 7 ring members), the ring can have carbon atoms and optionally one or more (e.g., 1 to 3) additional heteroatoms independently selected from nitrogen (N), oxygen (O), or sulfur (S). The ring can be saturated or partially saturated and can be optionally substituted.

For the purposed of the present invention fused ring units, as well as spirocyclic rings, bicyclic rings and the like, which comprise a single heteroatom will be considered to belong to the cyclic family corresponding to the heteroatom containing ring. For example, 1,2,3,4-tetrahydroquinoline having the formula:

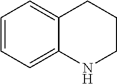

is, for the purposes of the present invention, considered a heterocyclic unit. 6,7-Dihydro-5H-cyclopentapyrimidine having the formula:

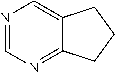

is, for the purposes of the present invention, considered a heteroaryl unit. When a fused ring unit contains heteroatoms in both a saturated and an aryl ring, the aryl ring will predominate and determine the type of category to which the ring is assigned. For example, 1,2,3,4-tetrahydro-[1,8]naphthyridine having the formula:

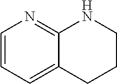

is, for the purposes of the present invention, considered a heteroaryl unit.

Whenever a term or either of their prefix roots appear in a name of a substituent the name is to be interpreted as including those limitations provided herein. For example, whenever the term “alkyl” or “aryl” or either of their prefix roots appear in a name of a substituent (e.g., arylalkyl, alkylamino) the name is to be interpreted as including those limitations given above for “alkyl” and “aryl.”

The term “substituted” is used throughout the specification. As used herein, the term “substituted” is defined as a moiety, whether acyclic or cyclic, which has one or more hydrogen atoms replaced by a substituent or several (e.g., 1 to 10) substituents as defined herein below. The substituents are capable of replacing one or two hydrogen atoms of a single moiety at a time. In addition, these substituents can replace two hydrogen atoms on two adjacent carbons to form said substituent, new moiety or unit. For example, a substituted unit that requires a single hydrogen atom replacement includes halogen, hydroxyl, and the like. A two hydrogen atom replacement includes carbonyl, oximino, and the like. A two hydrogen atom replacement from adjacent carbon atoms includes epoxy, and the like. The term “substituted” is used throughout the present specification to indicate that a moiety can have one or more of the hydrogen atoms replaced by a substituent. When a moiety is described as “substituted” any number of the hydrogen atoms may be replaced. For example, difluoromethyl is a substituted C₁ alkyl; trifluoromethyl is a substituted C₁ alkyl; 4-hydroxyphenyl is a substituted aromatic ring; (N,N-dimethyl-5-amino)octanyl is a substituted C₈ alkyl; 3-guanidinopropyl is a substituted C₃ alkyl; and 2-carboxypyridinyl is a substituted heteroaryl.

The variable groups defined herein, e.g., alkyl, alkenyl, alkynyl, cycloalkyl, alkoxy, aryloxy, aryl, heterocycle and heteroaryl groups defined herein, whether used alone or as part of another group, can be optionally substituted. Optionally substituted groups will be so indicated.

The following are non-limiting examples of substituents which can substitute for hydrogen atoms on a moiety: halogen (chlorine (Cl), bromine (Br), fluorine (F) and iodine(I)), —CN, —NO₂, oxo (═O), —OR⁶, —SR⁶, —N(R⁶)₂, —NR⁶C(O)R⁶, —SO₂R⁶, —SO₂OR⁶, —SO₂N(R⁶)₂, —C(O)R⁶, —C(O)OR⁶, —C(O)N(R⁶)₂, C₁₋₆ alkyl, C₁₋₆ haloalkyl, C₁₋₆ alkoxy, C₂₋₈ alkenyl, C₂₋₈ alkynyl, C₃₋₁₄ cycloalkyl, aryl, heterocycle, or heteroaryl, wherein each of the alkyl, haloalkyl, alkenyl, alkynyl, alkoxy, cycloalkyl, aryl, heterocycle, and heteroaryl groups is optionally substituted with 1-10 (e.g., 1-6 or 1-4) groups selected independently from halogen, —CN, —NO₂, oxo, and R⁶; wherein R⁶, at each occurrence, independently is hydrogen, —OR⁷, —SR⁷, —C(O)R⁷, —C(O)OR⁷, —C(O)N(R⁷)₂, —SO₂R⁷, —S(O)₂OR⁷, —N(R⁷)₂, —NR⁷C(O)R⁷, C₁₋₆ alkyl, C₁₋₆ haloalkyl, C₂₋₈ alkenyl, C₂₋₈ alkynyl, cycloalkyl (e.g., C₃₋₆ cycloalkyl), aryl, heterocycle, or heteroaryl, or two R⁶ units taken together with the atom(s) to which they are bound form an optionally substituted carbocycle or heterocycle wherein said carbocycle or heterocycle has 3 to 7 ring atoms; wherein R⁷, at each occurrence, independently is hydrogen, C₁₋₆ alkyl, C₁₋₆ haloalkyl, C₂₋₈ alkenyl, C₂₋₈ alkynyl, cycloalkyl (e.g., C₃₋₆ cycloalkyl), aryl, heterocycle, or heteroaryl, or two R⁷ units taken together with the atom(s) to which they are bound form an optionally substituted carbocycle or heterocycle wherein said carbocycle or heterocycle preferably has 3 to 7 ring atoms.

In some embodiments, the substituents are selected from
i) —OR⁸; for example, —OH, —OCH₃, —OCH₂CH₃, —OCH₂CH₂CH₃;
ii) —C(O)R⁸; for example, —COCH₃, —COCH₂CH₃, —COCH₂CH₂CH₃;
iii) —C(O)OR⁸; for example, —CO₂CH₃, —CO₂CH₂CH₃, —CO₂CH₂CH₂CH₃;
iv) —C(O)N(R⁸)₂; for example, —CONH₂, —CONHCH₃, —CON(CH₃)₂;
v) —N(R⁸)₂; for example, —NH₂, —NHCH₃, —N(CH₃)₂, —NH(CH₂CH₃);
vi) halogen: —F, —Cl, —Br, and —I;
vii) —CHₑX_{g}; wherein X is halogen, m is from 0 to 2, e+g=3; for example, —CH₂F, —CHF₂, —CF₃, —CCl₃, or —CBr₃;
viii) —SO₂R⁸; for example, —SO₂H; —SO₂CH₃; —SO₂C₆H₅;
ix) C₁-C₆ linear, branched, or cyclic alkyl;
x) Cyano
xi) Nitro;
xii) N(R⁸)C(O)R⁸;
xiii) Oxo (═O);
xiv) Heterocycle; and
xv) Heteroaryl;
   wherein each R⁸ is independently hydrogen, optionally substituted C₁-C₆ linear or branched alkyl (e.g., optionally substituted C₁-C₄ linear or branched alkyl), or optionally substituted C₃-C₆ cycloalkyl (e.g. optionally substituted C₃-C₄ cycloalkyl); or two R⁸ units can optionally be taken together to form a ring comprising 3-7 ring atoms. In certain aspects, each R⁸ is independently hydrogen, C₁-C₆ linear or branched alkyl optionally substituted with halogen or C₃-C₆ cycloalkyl or C₃-C₆ cycloalkyl.

At various places in the present specification, substituents of compounds are disclosed in groups or in ranges. It is specifically intended that the description include each and every individual subcombination of the members of such groups and ranges. For example, the term “C₁₋₆ alkyl” is specifically intended to individually disclose C₁, C₂, C₃, C₄, C₅, C₆, C₁-C₆, C₁-C₅, C₁-C₄, C₁-C₃, C₁-C₂, C₂-C₆, C₂-C₅, C₂-C₄, C₂-C₃, C₃-C₆, C₃-C₅, C₃-C₄, C₄-C₆, C₄-C₅, and C₅-C₆, alkyl.

For the purposes of the present invention the terms “compound,” “analog,” and “composition of matter” stand equally well for the compounds of the disclosure described herein, including all enantiomeric forms, diastereomeric forms, salts, and the like, and the terms “compound,” “analog,” and “composition of matter” are used interchangeably throughout the present specification.

When any variable occurs more than one time in any constituent or in any formula, its definition in each occurrence is independent of its definition at every other occurrence (e.g., in N(R⁷)₂, each R⁷ may be the same or different than the other). Combinations of substituents and/or variables are permissible only if such combinations result in stable compounds.

The terms “treat” and “treating” and “treatment” as used herein, refer to partially or completely alleviating, inhibiting, ameliorating and/or relieving a condition from which a patient is suspected to suffer.

As used herein, “therapeutically effective” and “effective dose” refer to a substance or an amount that elicits a desirable biological activity or effect.

Except when noted, the terms “subject” or “patient” are used interchangeably and refer to mammals such as human patients and non-human primates, as well as experimental animals such as rabbits, rats, and mice, and other animals. Accordingly, the term “subject” or “patient” as used herein means any mammalian patient or subject to which the compounds of the invention can be administered. In an exemplary embodiment of the present invention, to identify subject patients for treatment according to the methods of the invention, accepted screening methods are employed to determine risk factors associated with a targeted or suspected disease or condition or to determine the status of an existing disease or condition in a subject. These screening methods include, for example, conventional work-ups to determine risk factors that may be associated with the targeted or suspected disease or condition. These and other routine methods allow the clinician to select patients in need of therapy using the methods and compounds of the present invention.

Throughout this disclosure, various aspects of the invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 2.7, 3, 4, 5, 5.3, 6 and any whole and partial increments therebetween. This applies regardless of the breadth of the range.

### Description

The present invention relates to compositions and methods for treating lipid disorders in a subject. In one embodiment, the compositions of the present invention can be used to inhibit protease proprotein convertase subtilisin-like kexin type 9 (PCSK9). In another embodiment, the compositions of the present invention can be used to disrupt the protein-protein interaction between PCSK9 and the low-density lipoprotein receptor (LDLR).

### Compositions and Compounds

The present invention relates to compounds that will bind to PCSK9 and block its binding to the LDLR. The virtual screening methods and novel cell-based assays described herein have been integrated into a simple, efficient procedure to identify hit compounds that can ultimately be optimized to produce a drug for the treatment of hypercholesterolemia. A robust virtual screening method has been developed that employs a customized Schrödinger (http://www.schrodinger.com/) software suite, including LigPrep, Glide, Prime, Jaguar, QSite, Phase, Liaison, MacroModel and Canvas. A novel cell-based recombinant assay for screening inhibitors of the PCSK9/LDLR interaction has also been developed and validated. Using virtual screening methods, approximately 1,000,000 diverse, “drug-like” compounds and over 400K larger MW compounds (>500 amu) were screened, selected from over 8 million purchasable “drug-like” molecules and 13 million all purchaseable molecules in their respective ZINC databases. After visual inspection of several hundred of the top scoring 2,000 drug-like and 1,000 larger MW compounds, 179 screening compounds derived from both datasets were selected. In addition, over 300 drug-like compounds from a diverse compound collection were tested in the PCSK9/LDLR HEK293 recombinant cell based assay and have no effect on the PCSK9 synthesis processing and secretion. These compounds were also confirmed to upregulate the endogenously expressed LDLR in the HepG2 cells in situ. From these collective screening campaigns, eleven (SBC-110,424, SBC-110,433, SBC-115,017, SBC-115,048, SBC-115,076, SBC-110,032, SBC-110,033, SBC-110,034, SBC-110,035, SBC-110,036, and SBC-110,037) compounds consistently exhibited concentration dependent increase in the LDLR as comparable to control, with IC50s in the low uM range. In particular, the EC50's of SBC-110,424, SBC-110,433, and SBC-115,076 have an EC50 of 3.5, 1.6, and 3.1 μM, respectively. Compounds SBC-110,03x and SBC-110,03y are direct analogs, and are structurally related to compounds SBC-110,03z through SBC-110,03m which are also direct analogues of one another. In turn, compounds SBC-110,424 and SBC-110,433 are analogues and have structural similarities to SBC-110,032-037. A preliminary search of the scientific and patent literature indicates that none of these compounds has been previously identified as inhibitors of PCSK9/LDLR interaction.

In one aspect, the invention relates to a composition comprising a compound of Formula I:

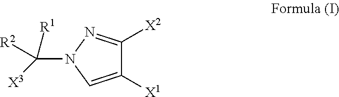

wherein in Formula (I):

R¹ is selected from the group consisting of hydrogen, C₁₋₆ alkyl, C₁₋₆ alkenyl, C₁₋₆ alkynyl, C₁₋₆ haloalkyl, optionally substituted aryl, and optionally substituted alkylaryl;

R² is selected from the group consisting of hydrogen, C₁₋₆ alkyl, C₁₋₆ alkenyl, C₁₋₆ alkynyl, C₁₋₆ haloalkyl, optionally substituted aryl, and optionally substituted alkylaryl, or R¹ and R² are optionally taken together with the atoms to which they are bound to form a ring containing 3 to 7 atoms, optionally containing a nitrogen, oxygen, or sulfur atom;

X¹ is selected from the group consisting of NR³COR⁴ and NR3SO₂R⁴

X² is selected from the group consisting of hydrogen and C₁₋₆ alkyl, or X¹ and X² are optionally taken together to form an optionally substituted aromatic six membered ring optionally containing a nitrogen atom;

X³ is selected from the group consisting of CONR³R⁴ and SO₂NR³R⁴;

R³ at each occurrence is independently selected from the group consisting of hydrogen and C₁₋₆ alkyl;

R⁴ at each occurrence is independently selected from the group consisting of hydrogen, C₁₋₆ alkyl, C₁₋₆ alkenyl, C₁₋₆ alkynyl, optionally substituted aryl, optionally substituted arylalkyl, optionally substituted heteroaryl, and optionally substituted heteroarylalkyl, or R³ and R⁴ are optionally taken together with the atoms to which they are bound to form a ring containing 3 to 7 atoms.

In one embodiment, R¹ and R² are methyl groups. In another embodiment, X¹ is NHCOR⁴. In another embodiment, R⁴ is selected from the group consisting of optionally substituted aryl, optionally substituted arylalkyl, optionally substituted heteroaryl, and optionally substituted heteroarylalkyl. In one embodiment, X³ is CONHCH₂R⁵ and R⁵ is selected from the group consisting of optionally substituted aryl, optionally substituted arylalkyl, optionally substituted heteroaryl, and optionally substituted heteroarylalkyl. In one embodiment, the composition of the invention comprises a compound of Formula (II):

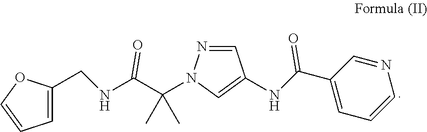 [SMILES: CC(C)(C(=O)NCc1ccco1)n1cc(NC(=O)c2cccnc2)cn1]

In another embodiment, the composition of the invention comprises a compound of Formula (III):

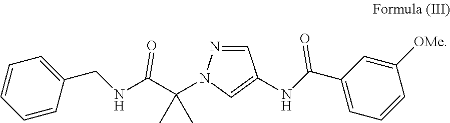 [SMILES: COc1cccc(C(=O)Nc2cnn(C(C)(C)C(=O)NCc3ccccc3)c2)c1]

In one aspect, the invention relates to a composition comprising a compound of Formula (IV):

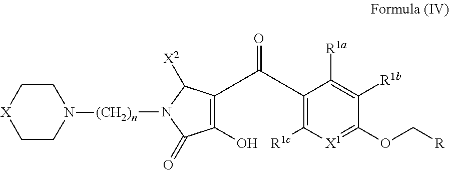

wherein in Formula (IV),

n is an integer from 1 to 6;

R is selected from the group consisting of optionally substituted aryl and optionally substituted heteroaryl;

R^{1a}, R^{1b}, R^{1c}, and R^{1d} are at each occurrence independently selected from the group consisting of hydrogen, halogen, optionally substituted C₁₋₆ alkyl, optionally substituted C₁₋₆ alkenyl, optionally substituted C₁₋₆ alkynyl, optionally substituted C₁₋₆ haloalkyl, optionally substituted C₃₋₇ cycloalkyl, optionally substituted C₁₋₆ alkoxy, cyano, nitro, OR², SR³, SO₂R³, NR^{4a}R^{4b}, NR^{4a}COR⁵, NR^{4a}CONR^{4a}R^{4b}, NR^{4a}COOR⁶, SO₂NR^{4a}R^{4b}, and NR^{4a}SO₂R⁶;

X is selected from the group consisting of oxygen, sulfur, NR^{4a}, NCOR⁵, NCONR^{4a}R^{4b}, NCOOR⁶, and NR^{4a}SO₂R³;

X¹ is selected from the group consisting of N and CR^{1d};

X² is selected from the group consisting of optionally substituted aryl and optionally substituted heteroaryl;

R² at each occurrence is independently selected from the group consisting of hydrogen, optionally substituted C₁₋₆ alkyl, optionally substituted C₁₋₆ alkenyl, optionally substituted C₁₋₆ alkynyl, optionally substituted C₁₋₆ haloalkyl, optionally substituted C₃₋₇ cycloalkyl, optionally substituted aryl, COR⁵, CONR^{4a}R^{4b}, SO₂NH₂, and SO₂R³;

R³ at each occurrence is independently selected from the group consisting of hydrogen, optionally substituted C₁₋₆ alkyl, optionally substituted C₁₋₆ alkenyl, optionally substituted C₁₋₆ alkynyl, optionally substituted C₁₋₆ haloalkyl, optionally substituted C₃₋₇ cycloalkyl, and optionally substituted aryl;

R^{4a} and R^{4b} at each occurrence are independently selected from the group consisting of hydrogen, optionally substituted C₁₋₆ alkyl, optionally substituted C₁₋₆ alkenyl, optionally substituted C₁₋₆ alkynyl, optionally substituted C₁₋₆ haloalkyl, optionally substituted C₃₋₇ cycloalkyl, and optionally substituted aryl;

R⁵ at each occurrence is independently selected from the group consisting of hydrogen, optionally substituted C₁₋₆ alkyl, optionally substituted C₁₋₆ alkenyl, optionally substituted C₁₋₆ alkynyl, optionally substituted C₁₋₆ haloalkyl, optionally substituted C₃₋₇ cycloalkyl, and optionally substituted aryl; and

R⁶ at each occurrence is independently selected from the group consisting of optionally substituted C₁₋₆ alkyl, optionally substituted C₁₋₆ alkenyl, optionally substituted C₁₋₆ alkynyl, optionally substituted C₁₋₆ haloalkyl, optionally substituted C₃₋₇ cycloalkyl, and optionally substituted aryl.

In one embodiment, n is 3. In another embodiment, X is oxygen. In one embodiment, X² is 4-pyridyl. In another embodiment, R is phenyl. In one embodiment, the composition of the invention comprises a compound of Formula (V):

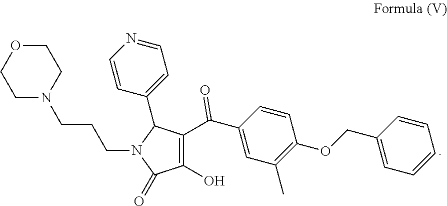 [SMILES: Cc1cc(C(=O)C2=C(O)C(=O)N(CCCN3CCOCC3)C2c2ccncc2)ccc1OCc1ccccc1]

Compounds described herein can contain an asymmetric atom (also referred as a chiral center), and some of the compounds can contain one or more asymmetric atoms or centers, which can thus give rise to optical isomers (enantiomers) and diastereomers. The present teachings and compounds disclosed herein include such enantiomers and diastereomers, as well as the racemic and resolved, enantiomerically pure R and S stereoisomers, as well as other mixtures of the R and S stereoisomers and pharmaceutically acceptable salts thereof. Optical isomers can be obtained in pure form by standard procedures known to those skilled in the art, which include, but are not limited to, diastereomeric salt formation, kinetic resolution, and asymmetric synthesis. The present teachings also encompass cis and trans isomers of compounds containing alkenyl moieties (e.g., alkenes and imines). It is also understood that the present teachings encompass all possible regioisomers, and mixtures thereof, which can be obtained in pure form by standard separation procedures known to those skilled in the art, and include, but are not limited to, column chromatography, thin-layer chromatography, and high-performance liquid chromatography.

### Methods of the Invention

In one aspect, the invention relates to a method for inhibiting proprotein convertase subtilisin-like kexin type 9 (PCSK9) in a subject. In one embodiment, the method comprises administering a therapeutically effective amount of a composition of the invention to a subject. In another aspect, the invention relates to a method for disrupting the protein-protein interaction between PCSK9 and low-density lipoprotein receptor (LDLR). In one embodiment, the method comprises administering a therapeutically effective amount of a composition of the invention to a subject. In one aspect, the invention relates to a method for treating a lipid disorder in a subject. In one embodiment, the method comprises administering a therapeutically effective amount of a composition of the invention to a subject. In another embodiment, the lipid disorder is hypercholesterolemia.

The establishment of a link between PCSK9 and cholesterol metabolism was rapidly followed by the discovery that selected mutations in the PCSK9 gene caused autosomal dominant hypercholesterolemia, suggesting that the mutations confer a gain-of-function by increasing the normal activity of PCSK9. This was supported by the experiment in which wild-type and mutant PCSK9 (S127R and F216L) were overexpressed in the livers of mice; hepatic LDLR protein levels fell dramatically in mice receiving either the wild-type or mutant PCSK9. No associated reductions in LDLR mRNA levels were observed, indicating that overexpression of PCSK9, whether mutant or wild-type, reduces LDLRs through a post-transcriptional mechanism.

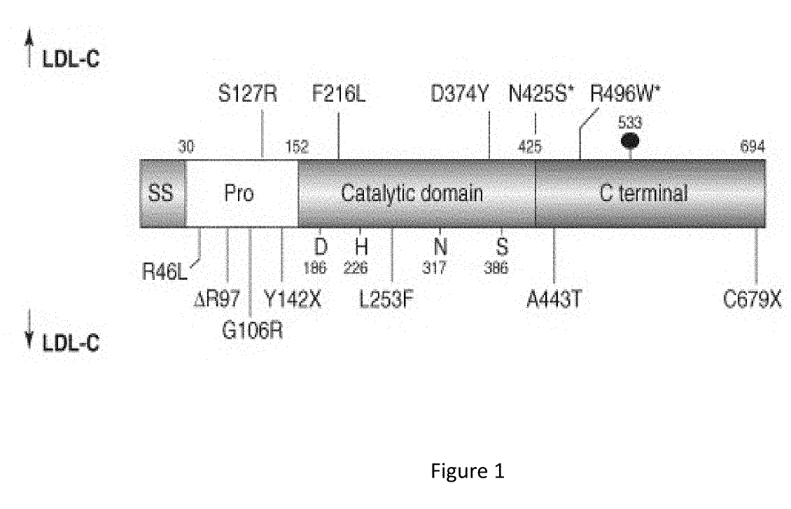

Given that gain-of-function mutations in PCSK9 causes hypercholesterolemia, loss-of-function mutations could have the opposite effect and result in hypocholesterolemia. Three loss-of-function mutations in PCSK9 (Y142X L253F and C679X) were identified in African-Americans. These mutations reduce LDL-C levels by 28% and were shown to decrease the frequency of CHD (defined as myocardial infarction, coronary death or coronary revascularization) by 88%. Rashid et al. studied the mechanism of loss-of-function mutations in mice where PCSK9 was inactivated. They reported that these knockout mice showed increased hepatic LDLR protein (but not mRNA), increased clearance of circulating lipoproteins and reduced plasma cholesterol levels. Structure-function relationship analysis of the naturally occurring mutations in PCSK9 has also provided insights into the mechanism of action of PCSK9. Interestingly, mutations in PCSK9 that were found to be associated with the greatest reductions in LDL-C plasma levels are those that prevent the secretion of mature PCSK9 by disrupting its synthesis (Y142X), autocatalytic processing (L253F) or folding (C679X). The Y142X mutation produces no detectable protein because it occurs early in the transcript and is predicted to initiate nonsense-mediated mRNA decay. Mutations in the catalytic domain (L253F) interfere with the autocatalytic cleavage of the protein. In cells expressing the PCSK9-253F, the amount of mature protein was reduced compared to that in cells expressing PCSK9-WT, suggesting that the mutation inhibits autocatalytic cleavage. The L253F mutation is near the catalytic triad (PCSK9 is a serine protease), therefore it might disrupt the active site function. Inasmuch as autocatalytic cleavage of PCSK9 is required for export of the protein out of the ER, the L253F mutation delays transport of PCSK9 from the ER to the cell surface. The nonsense mutation (C679X) in PCSK9, which truncates the protein by 14 amino acids at the C-terminus, did not interfere with protein processing, but the mature protein accumulates in the cells and is not secreted, suggesting that the protein is cleaved normally but is misfolded and is retained in the ER. FIG. 1 shows the location of the aforementioned mutations on the protein.

PCSK9 is a protease that belongs to the subtilisin family of kexin-like proconvertases. It contains a signal sequence of 30 amino acids followed by the prodomain of 122 amino acids, the catalytic domain, and a 279-amino acid cysteine- and histidine-rich C-terminal region known as the V domain. Unlike other proprotein convertases, the PCSK9 lacks a classical P domain that is required for folding and the regulation of protease activity.

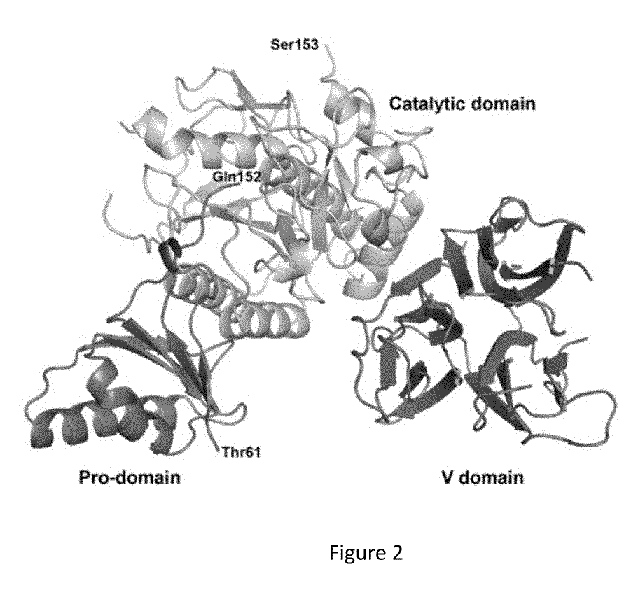

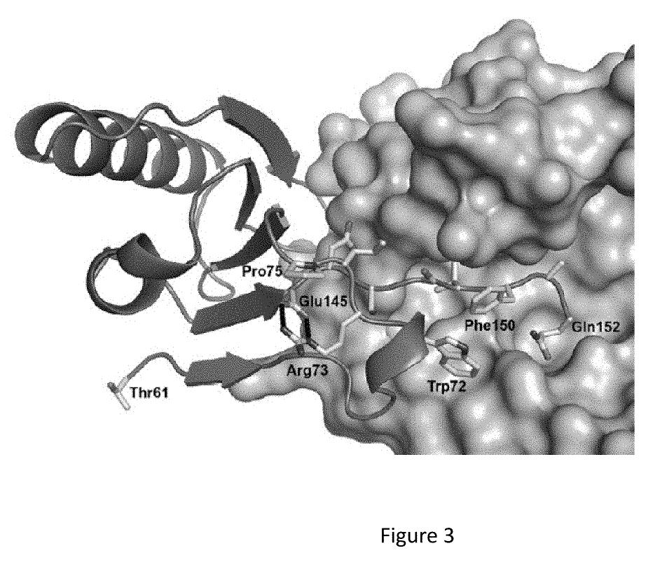

The protein is synthesized as a 72-kDa precursor that undergoes zymogen-processing between the prodomain and catalytic domain. The prodomain (14 kDa) remains bound to the mature protein (63 kDa) as it traverses the secretory pathway. The site of intramolecular cleavage in PCSK9 (Val-Phe-Ala-Gln↓Ser-Ile-Pro) differs from most other proconvertases in that cleavage does not occur after a basic residue. Obtaining a robust biochemical assay for PCSK9 activity has proved difficult and little is known about the requirements for catalytic activity. In contrast to other proprotein convertases, autocatalytic cleavage of PCSK9 does not require calcium. The mature PCSK9 and the associated prodomain both undergo tyrosine sulfation in the late Golgi complex before secretion. Sulfation of tyrosine residues in other proteins enhances protein-protein interactions, but the role of this post-translational modification in PCSK9 has not been defined. Recently, two different groups reported the high-resolution crystal structure of PCSK9. The crystal structure reveals that PCSK9 has subtilisin-like pro- and catalytic domains, and a V domain having a novel fold (FIG. 2). Although the full-length protein was crystallized, the crystal structure was found to be of the processed enzyme, with residues 152 and 153 separated by about 25 Å. The core of PCSK9's prodomain closely resembles the prodomain of subtilisin, with the 4 C-terminal residues of the prodomain bound in the active site forming an antiparallel b sheet with a strand from the catalytic domain (FIG. 3).

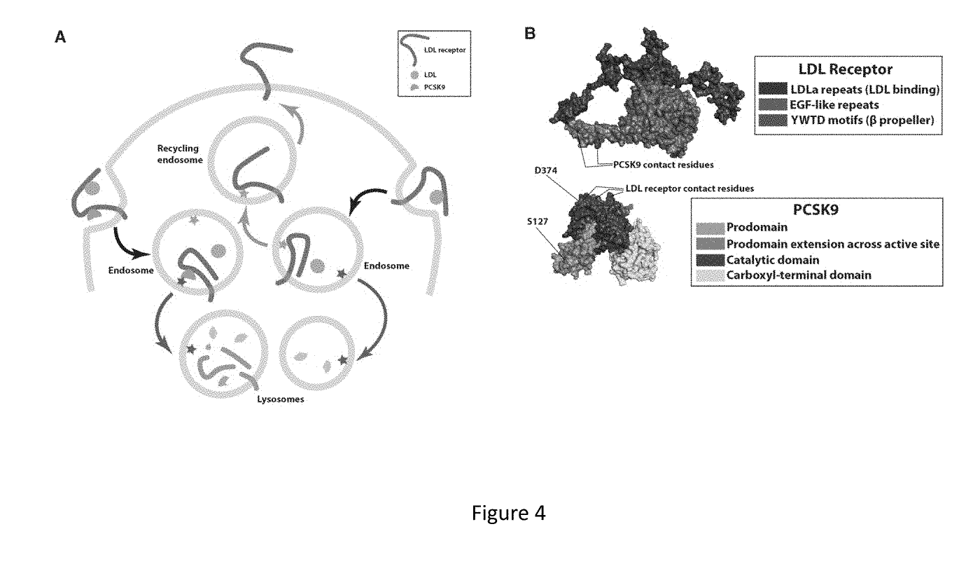

The LDLR is a multidomain protein that consists of a ligand binding domain, an epidermal growth factor (EGF) precursor homology domain, an O-glycosylated domain, a membrane spanning domain, and a cytoplasmic domain. Upon ligand binding to LDLR, the receptor/ligand complex is endocytosed, the ligand is released in the acidic environment of the endosome, and the LDLR is recycled to the cell surface. The ligand then undergoes lysosomal degradation. FIG. 4 is a diagrammatic representation of the possible pathway of LDLR/PCSK9 interaction.

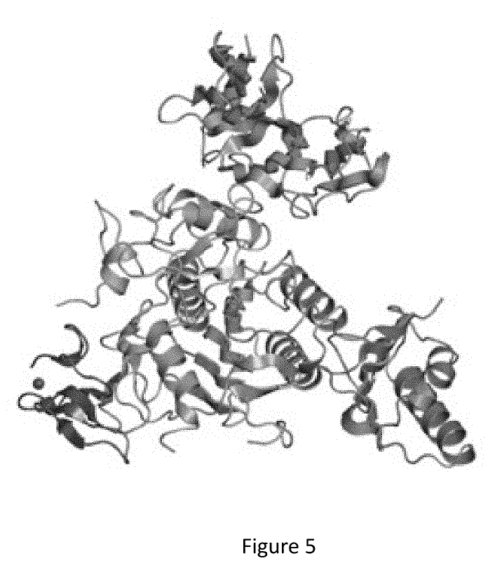

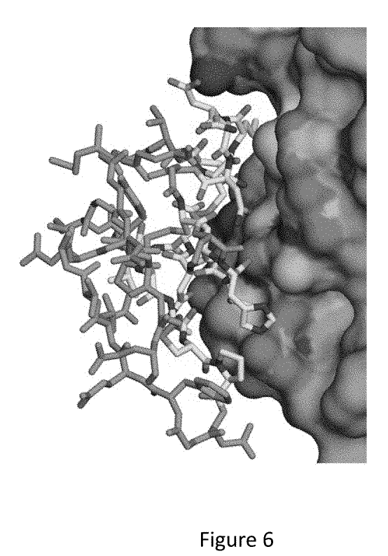

The mechanism by which PCSK9 causes the degradation of the LDLR has not been fully elucidated. However, it is clear that the protease activity of PCSK9 is not required for LDLR degradation. Li et al. have co-expressed the prodomain and the catalytic domain in trans, and showed that the secreted PCSK9 was catalytically inactive, yet it is functionally equivalent to the wild-type protein in lowering cellular LDL uptake and LDLR levels. Similar studies were also reported by McNutt et al. Furthermore, Zhang et al. have mapped PCSK9 binding to the EGF-A repeat of the LDLR, and showed that such binding decreases the receptor recycling and increases its degradation. They also reported that binding to EGF-A domain was calcium-dependent and increased dramatically with reduction in pH from 7 to 5.2. Recently, Kwon et al. determined the crystal structure of PCSK9 in complex with the LDLR-EGF-AB (EGF-A and EGF-B). The structure shows a well-defined EGF-A domain, but the EGF-B domain is disordered and absent from their electron density map. The EGF-A domain binds to the PCSK9 catalytic domain at a site distant from the catalytic site, and makes no contact with either the C-terminal domain or the prodomain (FIG. 5). The structure also maps the site of interaction between PCSK9 and the LDLR (FIG. 6), and therefore defines a potential therapeutic target site for blocking agents that could interfere with this interaction in vivo.

Several strategies have been proposed for targeting PCSK9. mRNA knockdown approaches include the use of antisense oligonucleotides or RNAi. Antisense oligonucleotides administered to mice reduced PCSK9 expression by >90% and lowered plasma cholesterol levels by 53%. A single intravenous injection of an RNAi delivered in lipidoid nanoparticles to cynomologous monkeys reduced plasma PCSK9 levels by 70% and plasma LDL-C levels by 56%. A second approach is to develop small-molecule inhibitors of the PCSK9 processing. Despite evidence that the catalytic activity of PCSK9 is not required for LDLR degradation, an intracellular inhibitor of PCSK9 catalytic activity should be effective, since autocatalytic processing of PCSK9 is required for secretion of the protein from the ER. A third approach, the one pursued in our proposed studies, is to prevent binding of PCSK9 to the LDLR on cell surface with small molecules, peptides, or antibodies directed against PCSK9. Adding EGF-A fragments to cultured cells inhibits the ability of exogenously added PCSK9 to mediate LDLR degradation. Recently Phase II proof-of-concept data have been reported that validate blocking PCSK9 mAb as a strategy for lowering LDL-C in patients not controlled on standard statin therapy. Therefore, PCSK9 acts as a secreted factor to cause LDLR degradation and a small-molecule inhibitor that interfere with the PCSK9/LDLR binding should upregulate the intracellular LDLR and decrease plasma LDL-C.

The present invention relates to disrupting protein-protein interaction, which is fundamentally different than inhibiting enzymes. In general, the active site of enzymes is a well-defined groove, while many of the surfaces involved in protein-protein interactions are rather flat. However, as can be seen from FIG. 6, the LDLR binding site on PCSK9 is not a flat surface. Furthermore, it is now clear that the bulk of the binding energy between two proteins is concentrated in a few “hotspots” that can potentially be disrupted with small molecules. Recently, Wells and McClendon reviewed successes in the discovery of small molecule antagonists of protein-protein interactions, and summarized data on six such examples. The molecular weight of antagonists of those six examples ranged from 400-900 Daltons. Therefore, in the virtual screening protocol described herein, a molecular weight cutoff is not imposed. A molecular weight cutoff of 500 Daltons is usually used when screening for enzyme inhibitors. It is also noteworthy that by using the virtual screening protocol, described herein, small molecule antagonists of the interface between the androgen receptor and its co-activator were able to be identified.

Compounds of the invention can be useful for the treatment or inhibition of a pathological condition or disorder in a mammal, for example, a human subject. The present teachings accordingly provide methods of treating or inhibiting a pathological condition or disorder by providing to a mammal a compound of the present teachings including its pharmaceutically acceptable salt) or a pharmaceutical composition that includes one or more compounds of the present teachings in combination or association with pharmaceutically acceptable carriers. Compounds of the present teachings can be administered alone or in combination with other therapeutically effective compounds or therapies for the treatment or inhibition of the pathological condition or disorder. In one embodiment, a compound of the invention can be administered in combination with an HMGCoA reductase inhibitor. In another embodiment, a compound of the invention can be administered in combination with a nicotinic acid. In another embodiment, a compound of the invention can be administered in combination with a fibric acid. In another embodiment, a compound of the invention can be administered in combination with a bile acid-binding resin.

### Formulations

The present invention also relates to compositions or formulations which comprise the compounds of the disclosure according to the present invention. In one embodiment, the formulation of the present invention comprises an effective amount of one or more compounds of the disclosure and salts thereof and one or more excipients.

For the purposes of the present invention the term “excipient” and “carrier” are used interchangeably throughout the description of the present invention and said terms are defined herein as, “ingredients which are used in the practice of formulating a safe and effective pharmaceutical composition.”

The formulator will understand that excipients are used primarily to serve in delivering a safe, stable, and functional pharmaceutical, serving not only as part of the overall vehicle for delivery but also as a means for achieving effective absorption by the recipient of the active ingredient. An excipient may fill a role as simple and direct as being an inert filler, or an excipient as used herein may be part of a pH stabilizing system or coating to insure delivery of the ingredients safely to the stomach. The formulator can also take advantage of the fact the compounds of the present invention have improved cellular potency, pharmacokinetic properties, as well as improved oral bioavailability.

The present teachings also provide pharmaceutical compositions that include at least one compound described herein and one or more pharmaceutically acceptable carriers, excipients, or diluents. Examples of such carriers are well known to those skilled in the art and can be prepared in accordance with acceptable pharmaceutical procedures, such as, for example, those described in Remington's Pharmaceutical Sciences, 17th edition, ed. Alfonoso R. Gennaro, Mack Publishing Company, Easton, Pa. (1985), the entire disclosure of which is incorporated by reference herein for all purposes. As used herein, “pharmaceutically acceptable” refers to a substance that is acceptable for use in pharmaceutical applications from a toxicological perspective and does not adversely interact with the active ingredient. Accordingly, pharmaceutically acceptable carriers are those that are compatible with the other ingredients in the formulation and are biologically acceptable. Supplementary active ingredients can also be incorporated into the pharmaceutical compositions.

Compounds of the present teachings can be administered orally or parenterally, neat or in combination with conventional pharmaceutical carriers. Applicable solid carriers can include one or more substances which can also act as flavoring agents, lubricants, solubilizers, suspending agents, fillers, glidants, compression aids, binders or tablet-disintegrating agents, or encapsulating materials. The compounds can be formulated in conventional manner, for example, in a manner similar to that used for known cholesterol lowering agents. Oral formulations containing a compound disclosed herein can comprise any conventionally used oral form, including tablets, capsules, buccal forms, troches, lozenges and oral liquids, suspensions or solutions. In powders, the carrier can be a finely divided solid, which is an admixture with a finely divided compound. In tablets, a compound disclosed herein can be mixed with a carrier having the necessary compression properties in suitable proportions and compacted in the shape and size desired. The powders and tablets can contain up to 99% of the compound.

Capsules can contain mixtures of one or more compound(s) disclosed herein with inert filler(s) and/or diluent(s) such as pharmaceutically acceptable starches (e.g., corn, potato or tapioca starch), sugars, artificial sweetening agents, powdered celluloses (e.g., crystalline and microcrystalline celluloses), flours, gelatins, gums, and the like.

Useful tablet formulations can be made by conventional compression, wet granulation or dry granulation methods and utilize pharmaceutically acceptable diluents, binding agents, lubricants, disintegrants, surface modifying agents (including surfactants), suspending or stabilizing agents, including, but not limited to, magnesium stearate, stearic acid, sodium lauryl sulfate, talc, sugars, lactose, dextrin, starch, gelatin, cellulose, methyl cellulose, microcrystalline cellulose, sodium carboxymethyl cellulose, carboxymethylcellulose calcium, polyvinylpyrrolidine, alginic acid, acacia gum, xanthan gum, sodium citrate, complex silicates, calcium carbonate, glycine, sucrose, sorbitol, dicalcium phosphate, calcium sulfate, lactose, kaolin, mannitol, sodium chloride, low melting waxes, and ion exchange resins. Surface modifying agents include nonionic and anionic surface modifying agents. Representative examples of surface modifying agents include, but are not limited to, poloxamer 188, benzalkonium chloride, calcium stearate, cetostearl alcohol, cetomacrogol emulsifying wax, sorbitan esters, colloidal silicon dioxide, phosphates, sodium dodecylsulfate, magnesium aluminum silicate, and triethanolamine. Oral formulations herein can utilize standard delay or time-release formulations to alter the absorption of the compound(s). The oral formulation can also consist of administering a compound disclosed herein in water or fruit juice, containing appropriate solubilizers or emulsifiers as needed.

Liquid carriers can be used in preparing solutions, suspensions, emulsions, syrups, elixirs, and for inhaled delivery. A compound of the present teachings can be dissolved or suspended in a pharmaceutically acceptable liquid carrier such as water, an organic solvent, or a mixture of both, or a pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers, and osmo-regulators. Examples of liquid carriers for oral and parenteral administration include, but are not limited to, water (particularly containing additives as described herein, e.g., cellulose derivatives such as a sodium carboxymethyl cellulose solution), alcohols (including monohydric alcohols and polyhydric alcohols, e.g., glycols) and their derivatives, and oils (e.g., fractionated coconut oil and arachis oil). For parenteral administration, the carrier can be an oily ester such as ethyl oleate and isopropyl myristate. Sterile liquid carriers are used in sterile liquid form compositions for parenteral administration. The liquid carrier for pressurized compositions can be halogenated hydrocarbon or other pharmaceutically acceptable propellants.

Liquid pharmaceutical compositions, which are sterile solutions or suspensions, can be utilized by, for example, intramuscular, intraperitoneal or subcutaneous injection. Sterile solutions can also be administered intravenously. Compositions for oral administration can be in either liquid or solid form.

Preferably the pharmaceutical composition is in unit dosage form, for example, as tablets, capsules, powders, solutions, suspensions, emulsions, granules, or suppositories. In such form, the pharmaceutical composition can be sub-divided in unit dose(s) containing appropriate quantities of the compound. The unit dosage forms can be packaged compositions, for example, packeted powders, vials, ampoules, prefilled syringes or sachets containing liquids. Alternatively, the unit dosage form can be a capsule or tablet itself, or it can be the appropriate number of any such compositions in package form. Such unit dosage form can contain from about 1 mg/kg of compound to about 500 mg/kg of compound, and can be given in a single dose or in two or more doses. Such doses can be administered in any manner useful in directing the compound(s) to the recipient's bloodstream, including orally, via implants, parenterally (including intravenous, intraperitoneal and subcutaneous injections), rectally, vaginally, and transdermally. Doses of the compound of the invention for administration may be in the range of from about 1 μg to about 10,000 mg, from about 20 μg to about 9,500 mg, from about 40 μg to about 9,000 mg, from about 75 μg to about 8,500 mg, from about 150 μg to about 7,500 mg, from about 200 μg to about 7,000 mg, from about 3050 μg to about 6,000 mg, from about 500 μg to about 5,000 mg, from about 750 μg to about 4,000 mg, from about 1 mg to about 3,000 mg, from about 10 mg to about 2,500 mg, from about 20 mg to about 2,000 mg, from about 25 mg to about 1,500 mg, from about 30 mg to about 1,000 mg, from about 40 mg to about 900 mg, from about 50 mg to about 800 mg, from about 60 mg to about 750 mg, from about 70 mg to about 600 mg, from about 80 mg to about 500 mg, and any and all whole or partial increments therebetween.

When administered for the treatment or inhibition of a particular disease state or disorder, it is understood that an effective dosage can vary depending upon the particular compound utilized, the mode of administration, and severity of the condition being treated, as well as the various physical factors related to the individual being treated. In therapeutic applications, a compound of the present teachings can be provided to a patient already suffering from a disease in an amount sufficient to cure or at least partially ameliorate the symptoms of the disease and its complications. The dosage to be used in the treatment of a specific individual typically must be subjectively determined by the attending physician. The variables involved include the specific condition and its state as well as the size, age and response pattern of the patient.

In some cases it may be desirable to administer a compound directly to the airways of the patient, using devices such as, but not limited to, metered dose inhalers, breath-operated inhalers, multidose dry-powder inhalers, pumps, squeeze-actuated nebulized spray dispensers, aerosol dispensers, and aerosol nebulizers. For administration by intranasal or intrabronchial inhalation, the compounds of the present teachings can be formulated into a liquid composition, a solid composition, or an aerosol composition. The liquid composition can include, by way of illustration, one or more compounds of the present teachings dissolved, partially dissolved, or suspended in one or more pharmaceutically acceptable solvents and can be administered by, for example, a pump or a squeeze-actuated nebulized spray dispenser. The solvents can be, for example, isotonic saline or bacteriostatic water. The solid composition can be, by way of illustration, a powder preparation including one or more compounds of the present teachings intermixed with lactose or other inert powders that are acceptable for intrabronchial use, and can be administered by, for example, an aerosol dispenser or a device that breaks or punctures a capsule encasing the solid composition and delivers the solid composition for inhalation. The aerosol composition can include, by way of illustration, one or more compounds of the present teachings, propellants, surfactants, and co-solvents, and can be administered by, for example, a metered device. The propellants can be a chlorofluorocarbon (CFC), a hydrofluoroalkane (HFA), or other propellants that are physiologically and environmentally acceptable.

Compounds described herein can be administered parenterally or intraperitoneally. Solutions or suspensions of these compounds or a pharmaceutically acceptable salts, hydrates, or esters thereof can be prepared in water suitably mixed with a surfactant such as hydroxyl-propylcellulose. Dispersions can also be prepared in glycerol, liquid polyethylene glycols, and mixtures thereof in oils. Under ordinary conditions of storage and use, these preparations typically contain a preservative to inhibit the growth of microorganisms.

The pharmaceutical forms suitable for injection can include sterile aqueous solutions or dispersions and sterile powders for the extemporaneous preparation of sterile injectable solutions or dispersions. In some embodiments, the form can sterile and its viscosity permits it to flow through a syringe. The form preferably is stable under the conditions of manufacture and storage and can be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), suitable mixtures thereof, and vegetable oils.

Compounds described herein can be administered transdermally, i.e., administered across the surface of the body and the inner linings of bodily passages including epithelial and mucosal tissues. Such administration can be carried out using the compounds of the present teachings including pharmaceutically acceptable salts, hydrates, or esters thereof, in lotions, creams, foams, patches, suspensions, solutions, and suppositories (rectal and vaginal).

Transdermal administration can be accomplished through the use of a transdermal patch containing a compound, such as a compound disclosed herein, and a carrier that can be inert to the compound, can be non-toxic to the skin, and can allow delivery of the compound for systemic absorption into the blood stream via the skin. The carrier can take any number of forms such as creams and ointments, pastes, gels, and occlusive devices. The creams and ointments can be viscous liquid or semisolid emulsions of either the oil-in-water or water-in-oil type. Pastes comprised of absorptive powders dispersed in petroleum or hydrophilic petroleum containing the compound can also be suitable. A variety of occlusive devices can be used to release the compound into the blood stream, such as a semi-permeable membrane covering a reservoir containing the compound with or without a carrier, or a matrix containing the compound. Other occlusive devices are known in the literature.

Compounds described herein can be administered rectally or vaginally in the form of a conventional suppository. Suppository formulations can be made from traditional materials, including cocoa butter, with or without the addition of waxes to alter the suppository's melting point, and glycerin. Water-soluble suppository bases, such as polyethylene glycols of various molecular weights, can also be used.

Lipid formulations or nanocapsules can be used to introduce compounds of the present teachings into host cells either in vitro or in vivo. Lipid formulations and nanocapsules can be prepared by methods known in the art.

To increase the effectiveness of compounds of the present teachings, it can be desirable to combine a compound with other agents effective in the treatment of the target disease. For example, other active compounds (i.e., other active ingredients or agents) effective in treating the target disease can be administered with compounds of the present teachings. The other agents can be administered at the same time or at different times than the compounds disclosed herein.

Pharmaceutically acceptable salts of compounds of the present teachings, which can have an acidic moiety, can be formed using organic and inorganic bases. Both mono and polyanionic salts are contemplated, depending on the number of acidic hydrogens available for deprotonation. Suitable salts formed with bases include metal salts, such as alkali metal or alkaline earth metal salts, for example sodium, potassium, or magnesium salts; ammonia salts and organic amine salts, such as those formed with morpholine, thiomorpholine, piperidine, pyrrolidine, a mono-, di- or tri-lower alkylamine (e.g., ethyl-tert-butyl-, diethyl-, diisopropyl-, triethyl-, tributyl- or dimethylpropylamine), or a mono-, di-, or trihydroxy lower alkylamine (e.g., mono-, di- or triethanolamine). Specific non-limiting examples of inorganic bases include NaHCO₃, Na₂CO₃, KHCO₃, K₂CO₃, Cs₂CO₃, LiOH, NaOH, KOH, NaH₂PO₄, Na₂HPO₄, and Na₃PO₄. Internal salts also can be formed. Similarly, when a compound disclosed herein contains a basic moiety, salts can be formed using organic and inorganic acids. For example, salts can be formed from the following acids: acetic, propionic, lactic, benzenesulfonic, benzoic, camphorsulfonic, citric, tartaric, succinic, dichloroacetic, ethenesulfonic, formic, fumaric, gluconic, glutamic, hippuric, hydrobromic, hydrochloric, isethionic, lactic, maleic, malic, malonic, mandelic, methanesulfonic, mucic, napthalenesulfonic, nitric, oxalic, pamoic, pantothenic, phosphoric, phthalic, propionic, succinic, sulfuric, tartaric, toluenesulfonic, and camphorsulfonic as well as other known pharmaceutically acceptable acids.

Non-limiting examples of compositions according to the present invention include from about 0.001 mg to about 1000 mg of one or more compounds of the disclosure according to the present invention and one or more excipients; from about 0.01 mg to about 100 mg of one or more compounds of the disclosure according to the present invention and one or more excipients; and from about 0.1 mg to about 10 mg of one or more compounds of the disclosure according to the present invention; and one or more excipients.

### Oral Administration

For oral administration, suitable forms include tablets, dragees, liquids, drops, suppositories, or capsules, caplets and gelcaps. The compositions formulated for oral use may be prepared according to any method known in the art and such compositions may contain one or more agents selected from the group consisting of inert, non-toxic pharmaceutically excipients that are suitable for the manufacture of tablets. Such excipients include, for example an inert diluent such as lactose; granulating and disintegrating agents such as cornstarch; binding agents such as starch; and lubricating agents such as magnesium stearate. The tablets may be uncoated or they may be coated by known techniques for elegance or to delay the release of the active ingredients. Formulations for oral use may also be presented as hard gelatin capsules wherein the active ingredient is mixed with an inert diluent.

For oral administration, the compounds of the invention may be in the form of tablets or capsules prepared by conventional means with pharmaceutically acceptable excipients such as binding agents (e.g., polyvinylpyrrolidone, hydroxypropylcellulose or hydroxypropylmethylcellulose); fillers (e.g., cornstarch, lactose, microcrystalline cellulose or calcium phosphate); lubricants (e.g., magnesium stearate, talc, or silica); disintegrates (e.g., sodium starch glycollate); or wetting agents (e.g., sodium lauryl sulphate). If desired, the tablets may be coated using suitable methods and coating materials such as OPADRY™ film coating systems available from Colorcon, West Point, Pa. (e.g., OPADRY™ OY Type, OYC Type, Organic Enteric OY-P Type, Aqueous Enteric OY-A Type, OY-PM Type and OPADRY™ White, 32K18400). Liquid preparation for oral administration may be in the form of solutions, syrups or suspensions. The liquid preparations may be prepared by conventional means with pharmaceutically acceptable additives such as suspending agents (e.g., sorbitol syrup, methyl cellulose or hydrogenated edible fats); emulsifying agent (e.g., lecithin or acacia); non-aqueous vehicles (e.g., almond oil, oily esters or ethyl alcohol); and preservatives (e.g., methyl or propyl p-hydroxy benzoates or sorbic acid).

Granulating techniques are well known in the pharmaceutical art for modifying starting powders or other particulate materials of an active ingredient. The powders are typically mixed with a binder material into larger permanent free-flowing agglomerates or granules referred to as a “granulation.” For example, solvent-using “wet” granulation processes are generally characterized in that the powders are combined with a binder material and moistened with water or an organic solvent under conditions resulting in the formation of a wet granulated mass from which the solvent must then be evaporated.

Melt granulation involves the use of materials that are solid or semi-solid at room temperature (i.e., having a relatively low softening or melting point range) to promote granulation of powdered or other materials, essentially in the absence of added water or other liquid solvents. The low melting solids, when heated to a temperature in the melting point range, liquefy to act as a binder or granulating medium. The liquefied solid spreads itself over the surface of powdered materials with which it is contacted, and on cooling, forms a solid granulated mass in which the initial materials are bound together. The resulting melt granulation may then be provided to a tablet press or be encapsulated for preparing the oral dosage form. Melt granulation improves the dissolution rate and bioavailability of an active (i.e., drug) by forming a solid dispersion or solid solution.

U.S. Pat. No. 5,169,645 discloses directly compressible wax-containing granules having improved flow properties. The granules are obtained when waxes are admixed in the melt with certain flow improving additives, followed by cooling and granulation of the admixture. In certain embodiments, only the wax itself melts in the melt combination of the wax(es) and additives(s), and in other cases both the wax(es) and the additives(s) melt.

The present invention also includes a multi-layer tablet comprising a layer providing for the delayed release of one or more compounds of the invention, and a further layer providing for the immediate release of a medication for treatment of G-protein receptor-related diseases or disorders. Using a wax/pH-sensitive polymer mix, a gastric insoluble composition may be obtained in which the active ingredient is entrapped, ensuring its delayed release.

### Parenteral Administration

For parenteral administration, the compounds of the invention may be formulated for injection or infusion, for example, intravenous, intramuscular or subcutaneous injection or infusion, or for administration in a bolus dose and/or continuous infusion. Suspensions, solutions or emulsions in an oily or aqueous vehicle, optionally containing other formulatory agents such as suspending, stabilizing and/or dispersing agents may be used.

### Additional Administration Forms

Additional dosage forms of this invention include dosage forms as described in U.S. Pat. Nos. 6,340,475; 6,488,962; 6,451,808; 5,972,389; 5,582,837; and 5,007,790. Additional dosage forms of this invention also include dosage forms as described in U.S. Patent Applications Nos. 20030147952; 20030104062; 20030104053; 20030044466; 20030039688; and 20020051820. Additional dosage forms of this invention also include dosage forms as described in PCT Applications Nos. WO 03/35041; WO 03/35040; WO 03/35029; WO 03/35177; WO 03/35039; WO 02/96404; WO 02/32416; WO 01/97783; WO 01/56544; WO 01/32217; WO 98/55107; WO 98/11879; WO 97/47285; WO 93/18755; and WO 90/11757.

### Controlled Release Formulations and Drug Delivery Systems

In one embodiment, the formulations of the present invention may be, but are not limited to, short-term, rapid-offset, as well as controlled, for example, sustained release, delayed release and pulsatile release formulations.

The term sustained release refers to a drug formulation that provides for gradual release of a drug over an extended period of time, and that may, although not necessarily, result in substantially constant blood levels of a drug over an extended time period. The period of time may be as long as a day, a week, or a month or more and should be a release which is longer that the same amount of agent administered in bolus form. The term delayed release is used herein in its conventional sense to refer to a drug formulation that provides for an initial release of the drug after some delay following drug administration and that mat, although not necessarily, includes a delay of from about 10 minutes up to about 12 hours.

For sustained release, the compounds may be formulated with a suitable polymer or hydrophobic material which provides sustained release properties to the compounds. As such, the compounds for use the method of the invention may be administered in the form of microparticles, for example, by injection or in the form of wafers or discs by implantation.

In one embodiment of the invention, the compounds of the invention are administered to a patient, alone or in combination with another pharmaceutical agent, using a sustained release formulation.

The term pulsatile release refers to a drug formulation that provides release of the drug in such a way as to produce pulsed plasma profiles of the drug after drug administration.

The term immediate release refers to a drug formulation that provides for release of the drug immediately after drug administration.

As used herein, short-term refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes and any or all whole or partial increments thereof after drug administration after drug administration.

As used herein, rapid-offset refers to any period of time up to and including about 8 hours, about 7 hours, about 6 hours, about 5 hours, about 4 hours, about 3 hours, about 2 hours, about 1 hour, about 40 minutes, about 20 minutes, or about 10 minutes, and any and all whole or partial increments thereof after drug administration.

### Methods of Making

The present invention further relates to a process for preparing the compounds of the disclosure.

Compounds of the present teachings can be prepared in accordance with the procedures outlined herein, from commercially available starting materials, compounds known in the literature, or readily prepared intermediates, by employing standard synthetic methods and procedures known to those skilled in the art. Standard synthetic methods and procedures for the preparation of organic molecules and functional group transformations and manipulations can be readily obtained from the relevant scientific literature or from standard textbooks in the field. It will be appreciated that where typical or preferred process conditions (i.e., reaction temperatures, times, mole ratios of reactants, solvents, pressures, etc.) are given, other process conditions can also be used unless otherwise stated. Optimum reaction conditions can vary with the particular reactants or solvent used, but such conditions can be determined by one skilled in the art by routine optimization procedures. Those skilled in the art of organic synthesis will recognize that the nature and order of the synthetic steps presented can be varied for the purpose of optimizing the formation of the compounds described herein.

The processes described herein can be monitored according to any suitable method known in the art. For example, product formation can be monitored by spectroscopic means, such as nuclear magnetic resonance spectroscopy (e.g., 1H or 13C), infrared spectroscopy, spectrophotometry (e.g., UV-visible), mass spectrometry, or by chromatography such as high pressure liquid chromatograpy (HPLC), gas chromatography (GC), gel-permeation chromatography (GPC), or thin layer chromatography (TLC).

Preparation of the compounds can involve protection and deprotection of various chemical groups. The need for protection and deprotection and the selection of appropriate protecting groups can be readily determined by one skilled in the art. The chemistry of protecting groups can be found, for example, in Greene et al., Protective Groups in Organic Synthesis, 2d. Ed. (Wiley & Sons, 1991), the entire disclosure of which is incorporated by reference herein for all purposes.

The reactions or the processes described herein can be carried out in suitable solvents which can be readily selected by one skilled in the art of organic synthesis. Suitable solvents typically are substantially nonreactive with the reactants, intermediates, and/or products at the temperatures at which the reactions are carried out, i.e., temperatures that can range from the solvent's freezing temperature to the solvent's boiling temperature. A given reaction can be carried out in one solvent or a mixture of more than one solvent. Depending on the particular reaction step, suitable solvents for a particular reaction step can be selected.

The following series of diagrams provide non-limiting examples of methods of making compounds of the present invention:

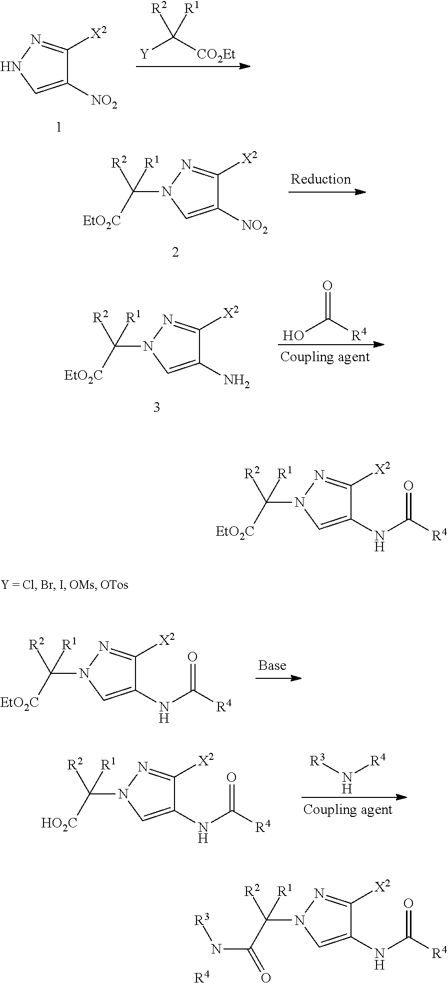

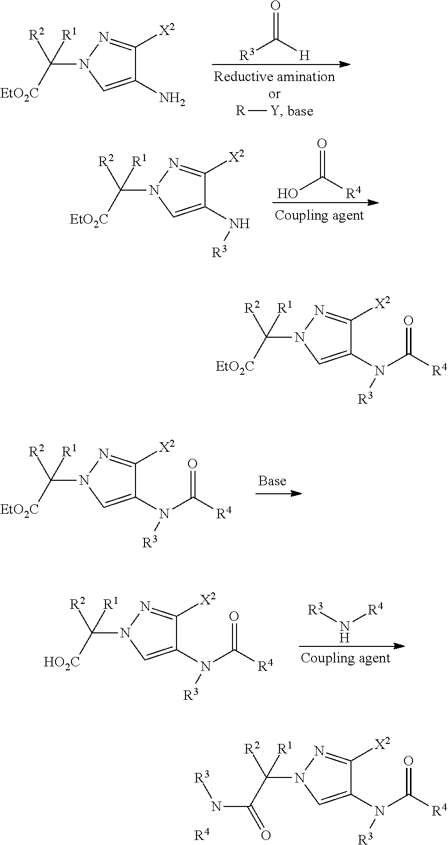

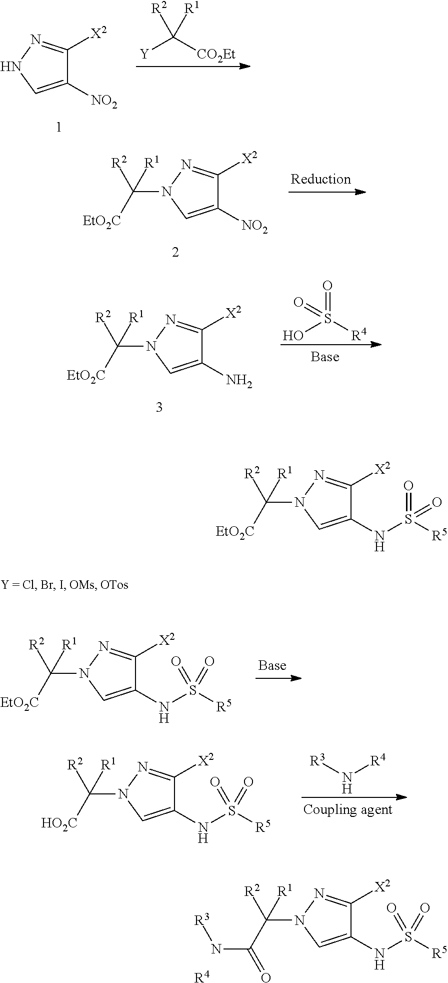

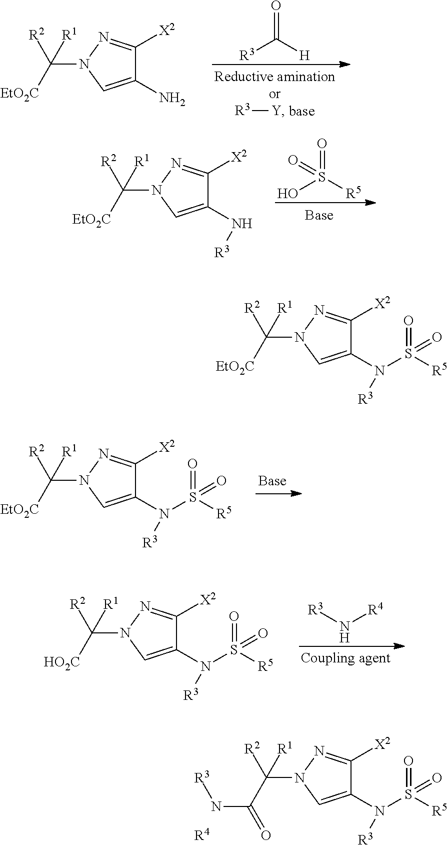

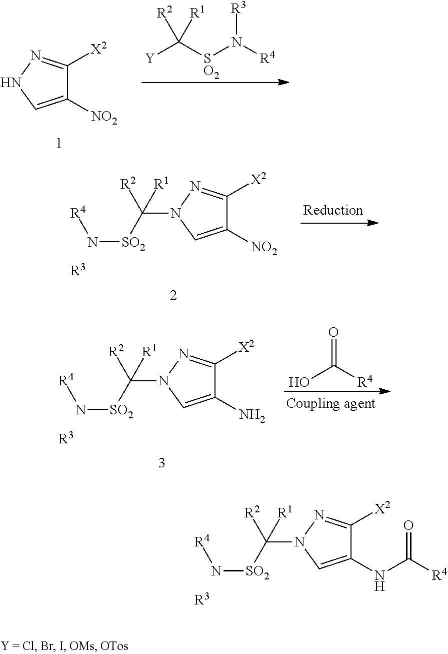

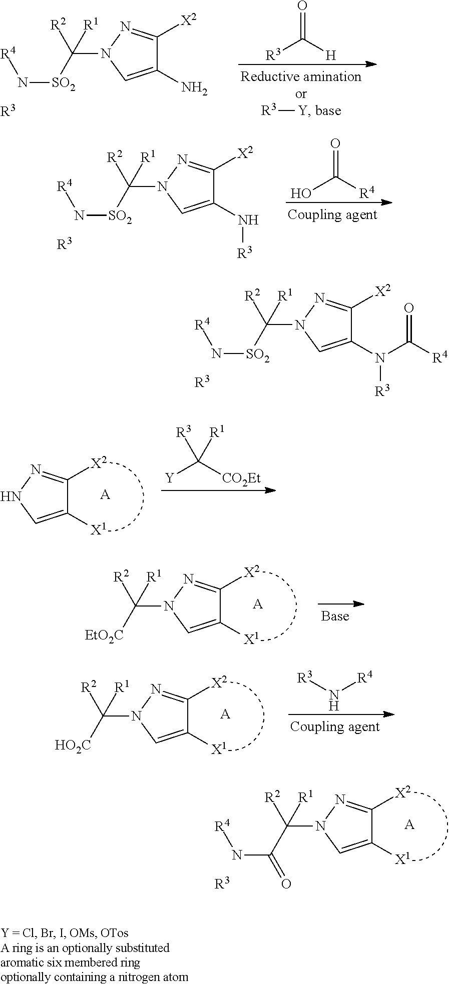

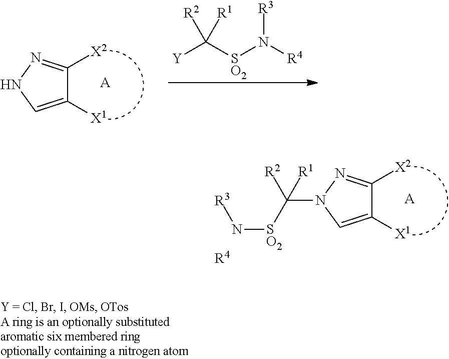

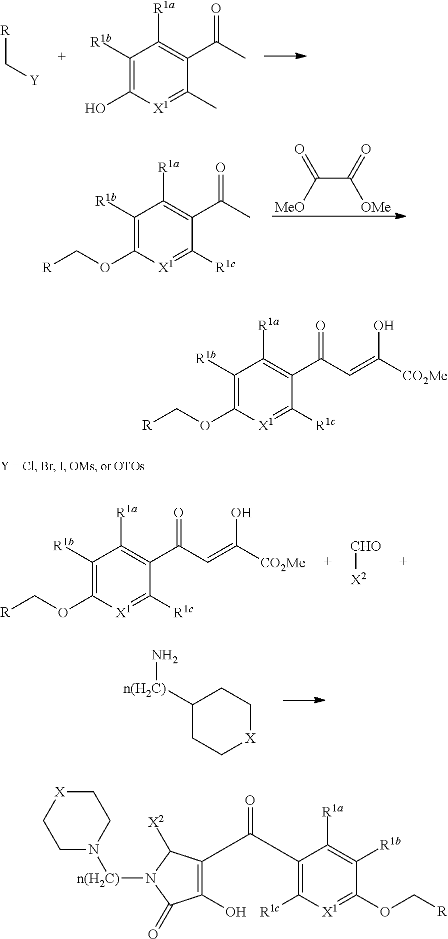

### EXPERIMENTAL EXAMPLES

The invention is further described in detail by reference to the following experimental examples. These examples are provided for purposes of illustration only, and are not intended to be limiting unless otherwise specified. Thus, the invention should in no way be construed as being limited to the following examples, but rather, should be construed to encompass any and all variations which become evident as a result of the teaching provided herein.

Without further description, it is believed that one of ordinary skill in the art can, using the preceding description and the following illustrative examples, make and utilize the compositions of the present invention and practice the claimed methods. The following working examples therefore, specifically point out the preferred embodiments of the present invention, and are not to be construed as limiting in any way the remainder of the disclosure.

### Example 1

### Use of Virtual Screening Methods to Identify Compounds that Bind to PCSK9 and Block its Binding to the LDLR

### Preparation of Compound Libraries for Docking Studies

The free ZINC database of commercially-available compounds for virtual screening as developed by Dr. Brian Shoichet of the University of California, San Francisco was identified as a starting point for compound selection. The “drug-like” database of over 8M purchaseable compounds (as of 2009) from the ZINC website (http://zinc.docking.org/) was used. A diverse subset of approx. 1M compounds was selected from the drug-like set using MACCS fingerprints and a Tanimoto similarity criterion of 0.90, i.e., every compound in the ˜1M set was less than 0.90 similar to any other compound. In addition, approximately 410,000 compounds from the ZINC all-purchaseable database (over 13 M compounds as of 2009) were selected with a MW cutoff of >500 amu's in order to include larger molecules potentially better capable of disrupting the protein-protein interaction between PCSK9 and its receptor. The Schrodinger program, LigPrep was used to convert the compounds from 2D to 3D for further computational analyses, including virtual screening, and it also adjusts ionization states to physiological conditions. As LigPrep also generates many chiral variants and some ring conformations, the resulting dataset size was ˜2.4M ligands for the drug-like set and ˜1M ligands for the >500 MW set.

### Defining the Site on the Protein for Compound Docking

The crystal structure of PDB ID 2P4E was used as the receptor structure for docking. The pro and V domains and all water molecules were removed (i.e., only the catalytic domain remained). Compounds were docked at the PCSK9 surface where the LDLR (EGF-A domain) binds. The region for docking was defined by the center of mass of the residue Phe379 of the PCSK9 chain. From visual inspection, this is roughly the central point of the main interaction area of the EGF-A domain with PCSK9. For the drug-like ligands set, ligands were constrained to be within a box of 18 Angstrom's centered on the Phe379 residue, whereas ligands from the >500 MW set were constrained to be within a box of 20 Angstrom's from this central point (to account for the larger size of the >500 MW ligands).

### Docking Studies

For docking, a customized Schrödinger (http://www.schrodinger.com/) virtual screening software suite was used, including LigPrep, Glide, Prime, Jaguar, QSite, Phase, Liaison, MacroModel and Canvas. The drug-like data set, due to its large size, was docked in parallel using the Glide program by splitting the dataset into 8 files. Compounds were docked in an automated and iterative fashion, initially using the high throughput virtual screening (HTVS) mode, and the highest scoring 10% ligands were re-docked using the standard precision (SP) scoring function, and finally the top 10% of those compounds were re-docked using the highest accuracy, lowest throughput XP scoring function, such that the final XP-output represented 1% of the total output of each docked set. The final merged XP output file for the drug-like set contained ˜24,000 compounds (˜3,000 from each file). The same procedure was used for the >500 MW subset resulting in a final total of ˜12,000 compounds (˜1,200 from each file). The Schrodinger Induced Fit Docking (IFD) protocol was applied to the final ˜24,000 and ˜12,000 compound sets separately. The IFD protocol accounts for receptor flexibility by allowing for receptor sidechain conformational change for those residues abutting and/or contacting the docked ligand. However, it is very computationally intensive compared to Glide docking (with a rigid protein) so IFD was only used with the top-scoring set of compounds from the Glide output and was only feasible due to access to a supercomputer cluster and the ability to run IFD in parallel. The top 2,000 compounds from IFD were retained for the drug-like dataset while only the top 1,000 compounds were retained from the >500 MW dataset.

### Compound Selection

Compounds with the highest IFD score (i.e., most negative) were presumed to have the best fit. As with all scoring functions, one cannot totally rely on the rank order. Therefore, the mode of binding of the top-scoring several hundred ligands from both the drug-like and >500 MW datasets were visually inspected. All ligands had a significant portion of their structure making good contact with the protein, so none were eliminated based on this criteria alone. Medicinal chemistry criteria were also utilized in the selection process. For example, compounds with potentially reactive functional groups were not selected. Emphasis was also placed on selecting a diverse set of chemotypes that fulfilled the aforementioned criteria. Finally, cost effective and reliable vendors were other key criteria used to purchase top-scoring compounds. Using these criteria, 179 compounds were selected and obtained for screening in an in vitro assay.

### Example 2

### Ability of Selected Compounds to Bind to PCSK9 and Prevent its Binding to the LDLR

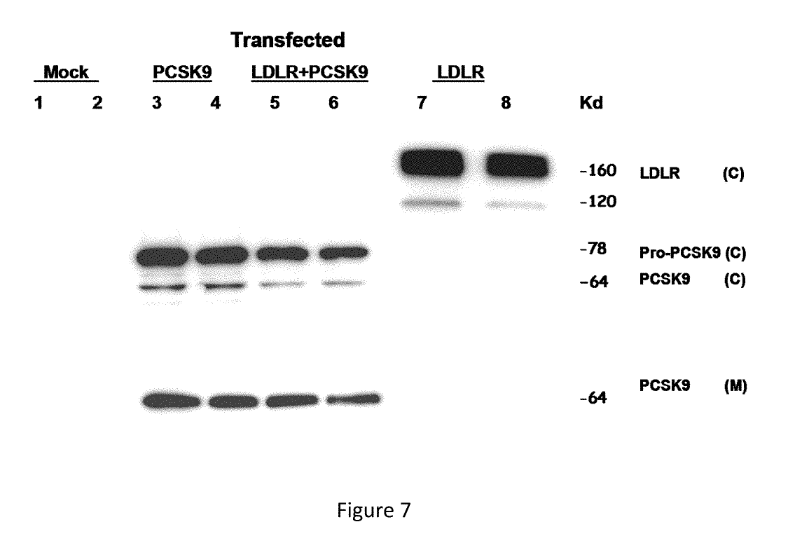

Considering the difficulty in assaying the LDLR/PCSK9 binding, the approach was to develop and implement an innovative recombinant cell based assay where the full-length PCSK9 and the LDLR genes are expressed into HEK-293 cells, and allowed to decrease the LDLR level by PCSK9 (FIG. 7). Compounds that prevent the LDLR degradation without preventing the auto-processing and secretion of the PCSK9 were selected as potential candidates and were further evaluated using additional cell based assay using endogenously expressed LDLR in liver cells (HepG2 cells).

### Development of a Novel Cell Based Assay for LDLR and PCSK9 Interaction.

Preparation of constructs: the human full-length PCSK9 was cloned. The cDNA encoding the PCSK9 sequence with and without a FLAG epitope tag (DYKDDDDK) were cloned by PCR and subcloned into mammalian expression vector containing the cytomegalovirus promoter-enhancer (pCMV-PCSK9-FLAG) (pCDNA3.1/PCSK9). For LDLR, the coding sequence was constructed without tag in mammalian expression vector (pCMV-LDLR) (pCDNA3.1/LDLR). The constructs were confirmed by sequence analysis

Analysis of PCSK9 expression in mammalian cells: The above constructs were used to transfect HEK-293 cells for expression. HEK-293T cells were seeded into 48-well plates in a DMEM containing 10% Fetal Bovine Serum media and incubated overnight at 37° C. Cells were transiently transfected with PCSK9 cDNA constructs using the Lipofectamine-LTX as described by the manufactures (Invitrogen). After transfection, cells were then incubated for additional 72 hours at 37° C., and replaced with serum free DMEM media containing ITS. Cells were lysed and media were collected and precipitated with TCA and analyzed by western blots as described below and in the caption of FIG. 7. The data (FIG. 7) showed that the PCSK9 is made as a pro-PCSK9 with MW around 74 K, which is processed to a 64 K mature protein. The mature protein is secreted into the media.

Analysis of LDLR/PCSK9 interaction: An assay was developed to demonstrate that expression of PCSK9 in HEK-293 cells decreases the expression level of the LDLR. The pCDNA3.1/PCSK9, and the pCDNA3.1/LDLR constructs were used to transiently transfect mammalian cells. The human embryonic kidney (HEK)-293 cells were seeded (1×105 cells/well) in 24-well plates. Expression plasmids (1 mg/well) were transiently transfected into HEK-293 cells with lipofectamine (Invitrogen) according to the manufacturer's protocol. Twenty four to forty eight hours after transfection, cells were harvested. The cells were then be lysed. Both cell lysate and supernatant were subjected to SDS-PAGE and immunoblot analysis using an anti-PCSK9 or LDLR antibody. The data from the blot showed that cells that were transfected with PCSK9 expressed both the unprocessed and the processed PCSK9 (cells), and processed PCSK9 (media). Cells that were transfected with LDLR showed expression of the LDLR in the cells. However, cells that were transfected with both LDLR and PCSK9 resulted in the disappearance of the LDLR in the cell lysate which indicate that the PCSK9 expression results in the degradation of the LDLR (FIG. 7).

Cell viability assay: For cell viability, 100 μl DMEM media containing the fluorogenic substrate Resazurin (Sigma 199303) were added to the HEK293 cells, incubated for 2 hrs, and quantitated using the fluorescence PerkinElmer Envision 2101 Multi-label plate reader with Excitation, Bodipy TMR FP 531 and Emission Rohdamine 590.

### Compound Screening

240 compounds identified from the virtual screen in a cell based assay for their potential to upregulate the LDLR in the presence of PCSK9 were tested as described above. Initial screening was done using 50 μM of the compounds. Each compound was added to the cell media in triplicates, and intrecellar LDLR was detected as described above. Each set of compounds were screened at least twice with all appropriate controls. Compounds that upregulate the LDLR were selected for further evaluation.

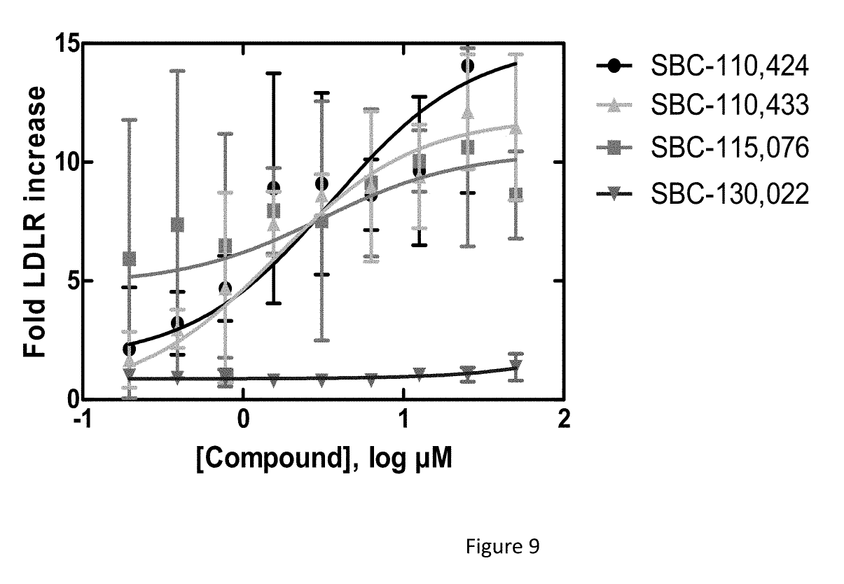

From the initial screening, 24 compounds were identified as potential hits. Hits confirmation was repeated in triplicates using 50 μM. Compounds that showed cell toxicity were excluded. Compounds that showed high potency were selected and were tested using various concentration of each compound ranging from 1-50 μM. FIG. 9 shows the effect of various concentration of the ten most potent compounds on the intracellular recombinant LDLR upregulation. SBC-110,424, SBC-110,433, SBC-115,017, SBC-115,048, SBC-115,076, SBC-110,032, SBC-110,033, SBC-110,034, SBC-110,035, SBC-110,036, and SBC-110,037 exhibited concentration dependent increase in the LDLR as comparable to control. FIG. 9 shows the concentration curve of three of these compounds (SBC-110,424, SBC-110,433 and SBC-115,076) to have an EC50 of 3.5, 1.6 and, 3.1 μM, respectively.

The compounds SBC-110,424, SBC-110, 433, and SBC-115,076 were also validated for their ability to up-regulate the endogenously expressed LDLR in HepG2 cells. The data shows that the three compounds exhibited a concentration dependent up-regulation of LDLR with an EC50 of 2.0, 2.9, and 0.43 μM.

### Mechanism of Action of the Compounds

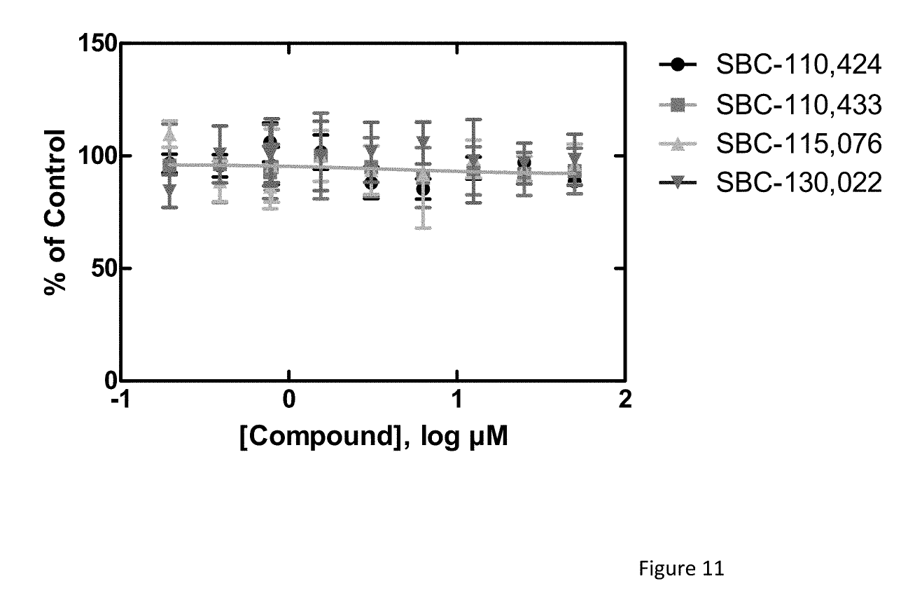

Increase degradation of LDLR by PCSK9: The ultimate goal is to identify compounds that bind to PCSK9 and block its binding to the LDLR. The increase in the level of recombinant LDLR in the presence of PCSK9 by the above hits could be either due to inhibiting the binding of the PCSK9 to the LDLR or by inhibiting the processing and secretion of the PCSK9. To eliminate the later possibility we tested the effect of these compounds on the processing and secretion of the PCSK9 as described below. The eleven screening hits were evaluated for inhibiting the processing and secretion of PCSK9. Cells were treated with each of these compounds (50-1 μM), then cell media and cells were analyzed by western blot. FIG. 11 shows the effect of these hits (only 4 are shown with similar effect for all other hits “data not shown”) on the processing and secretion of PCSK9 in the media. All the selected compounds exhibited no effect on the processing and secretion of the PCSK9 either in the cells or into the media. This supports an initial hypothesis that these compounds does not disrupt its synthesis, processing, and does not affect the amount of the mature secreted PCSK9.

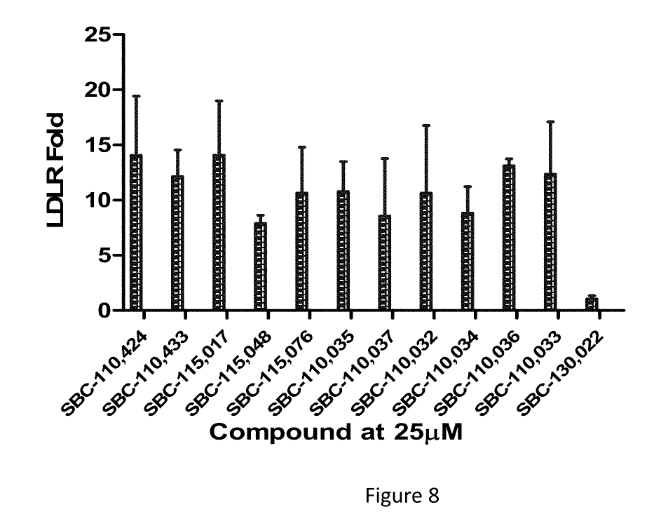

Screening Hits: As indicated above, eleven screening hits have been identified. These compounds exhibited concentration dependent increase in LDLR level as comparable to control. SBC-110,424 and SBC-110,433 are analogues and displayed significant potency for LDLR upregulation. Notably, these compounds are chiral compounds (FIG. 8) and were screened as racemates, so one of the two enantiomers should be at least 2-fold more potent should it be tested as pure isomer.

### Example 3

### Structure Activity Relationships (SAR) and Lead Optimization

Evidence of SAR can be indicated by small changes in the structure of a molecule resulting in small changes in its biological activity, or large structural changes resulting in large changes in activity. The process of SAR development and lead optimization is an iterative process that involves compound design, chemical synthesis and biological testing. Compound design is facilitated and accelerated by access to the crystal structure of PCSK9.

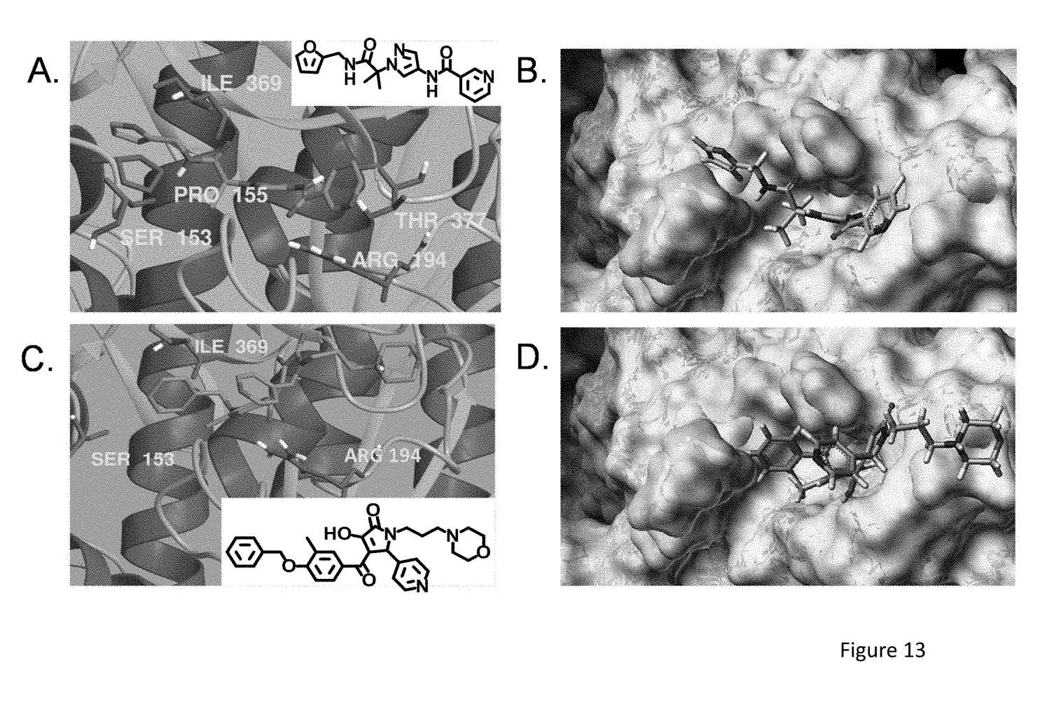

Experimental Design and Methods: Design criteria for chemical synthesis of analogues can employ available crystal structures. The choice of ligands for synthesis can be guided by synthetic feasibility and commercial availability and cost of building blocks. Compounds for purchase can be identified by substructure and similarity searches of commercial databases. The pyrazoles represented by SBC-110424 and the 4-hydroxy-5-oxopyrroles, represented by SBC-115,076, are the two most potent series identified herein (FIG. 13). Docking studies suggest that the binding sites for these two series partially overlap (FIG. 13). The goal of the medicinal chemistry effort is to identify lead molecules within the two series that display potency, selectivity and physicochemical/ADME properties that support their examination in animal efficacy models. The potency of the initial hits may need to be improved by 1-2 orders of magnitude in the LDL up-regulation assay to arrive at compounds that are suitable for in vivo studies. Analysis of the PCSK9 crystal structure suggests that the common point mutations seen in autosomal dominant hypercholesterolemia are not involved in the binding of the screening hits, nor are they positioned to influence the conformation of the binding sites that the screening hits occupy. Therefore, it is reasonable to assume that the screening hits and derived lead molecules would likely bind to these mutated PCSK9 proteins with similar affinity to that seen for normal PCSK9.

### Pyrazole Analogs Lead Optimization

Docking studies suggest that the pyrazole core and the furan moiety anchor SBC-110,424 in its binding site, with additional interactions between the protein and the two amide groups contributing to the binding (FIG. 13, panels A and B). The pyridyl moiety appears to lie along a solvent-exposed shelf of the protein surface and does not form strong interactions with the protein. The germinal dimethyl group adjacent to the pyrazole core lies at the entrance to a reasonably defined cleft in the protein surface. The present SAR strategy includes plans to functionalize this site on the molecule to take advantage of this cleft and provide additional interactions. The furan appears to be an important component of the binding of SBC-110,424, with both hydrogen bonding and lipophilic interactions present. However, furans are often toxic or are metabolized to toxic species. Therefore, a primary goal is to identify and refine bioisosteric replacements for the furan moiety that maintain similar hydrogen bonding and lipophilic characteristics such as benzofurans, thiophenes, oxazoles, isoxazoles, pyridines, substituted phenyl groups and oxime ethers (by way of non-limiting examples). Docking with other hits suggest that fusing an additional aryl or heteroaryl group onto the furan moiety should pick up additional interactions, so fused bicyclic substituents can be well represented in the diversity set. Iterative series of design, synthesis, testing and docking of structurally modified analogs can be pursued to refine the computational model.

The first phase of the medicinal chemistry campaign in the pyrazole series is to expand the SAR of the two pendant aromatic groups. While the furan SAR is the primary focus, it is important to confirm the role for the pyridine by preparing at least one series of analogs in that position. In addition, extension of the distance between this aromatic group and the proximal amide group might allow it to assume a conformation where it lies in a nearby shallow cleft on the protein, which might result in additional binding interactions.

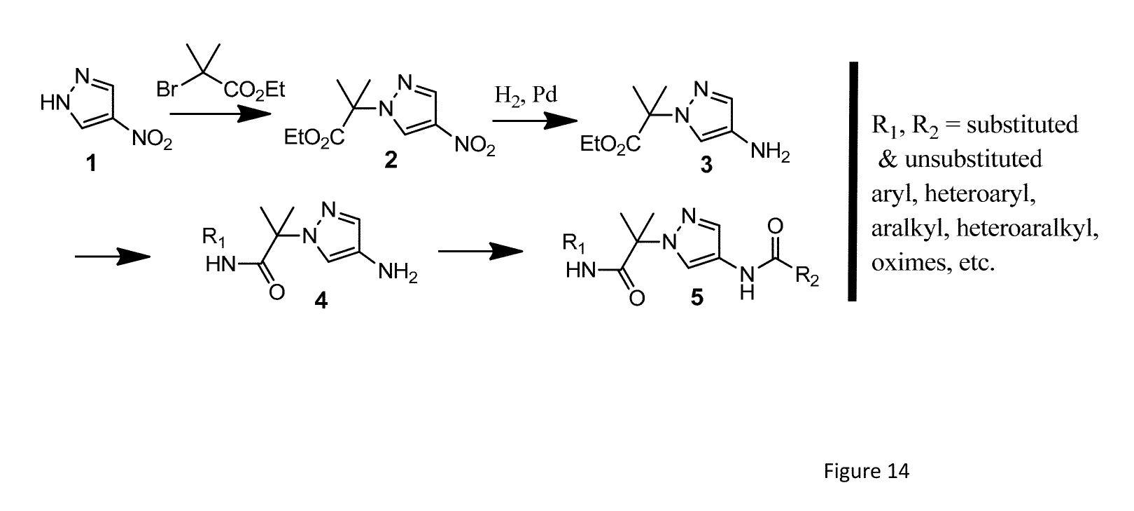

These types of analogs (compounds 5, FIG. 14) can be readily prepared through a common chemical strategy that is amenable to parallel synthesis. Catalytic hydrogenation is performed early in the synthesis to avoid complications with reductively-labile substituents.

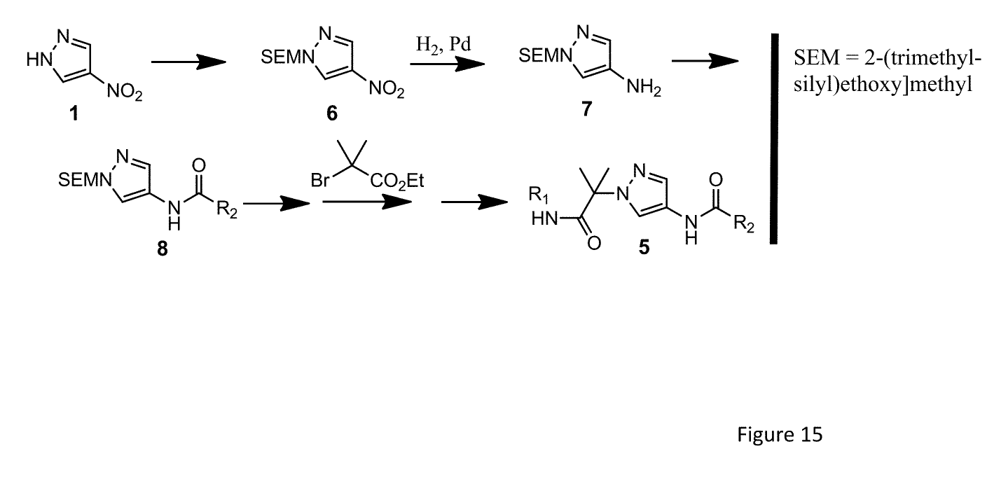

If difficulty is encountered in this synthetic scheme, an alternate route using the 2-(trimethylsilyl)ethoxylmethyl (SEM) protecting group can be used (FIG. 15).

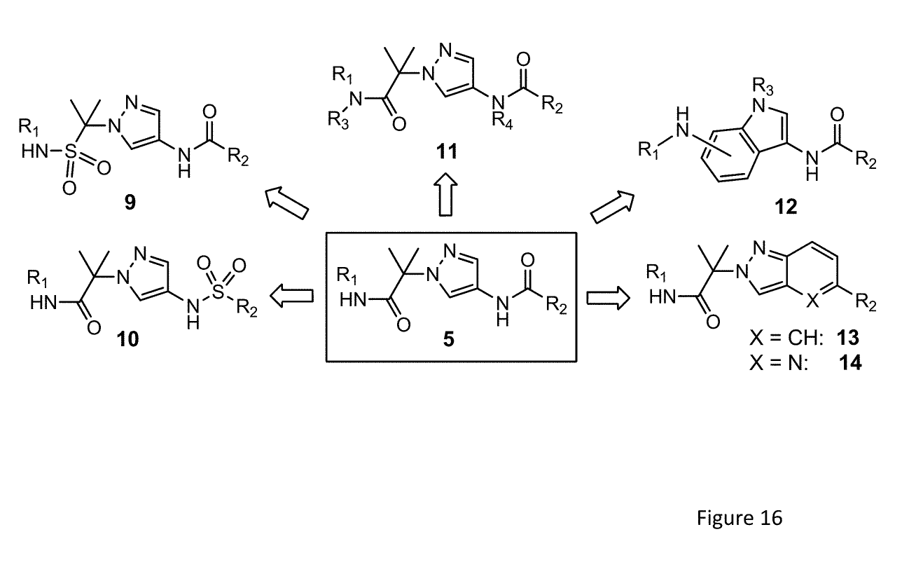

A second focus of the SAR on the pyrazole series is the two amide groups, which may be metabolically labile. We will synthesize analogs possessing more stable bioisosteric replacements (FIG. 16) such as sulfonamides (compounds 9,10) and N-alkylamines (compounds 11) as well as conformationally constrained bicyclic analogs such as indoles (compounds 12), 2H-indazoles (compounds 13) and pyrrazolopyridines (compounds 14). Data and iterative docking studies will be employed to refine the SAR (and the model) and design additional analogs that interact more efficiently with PCSK9.

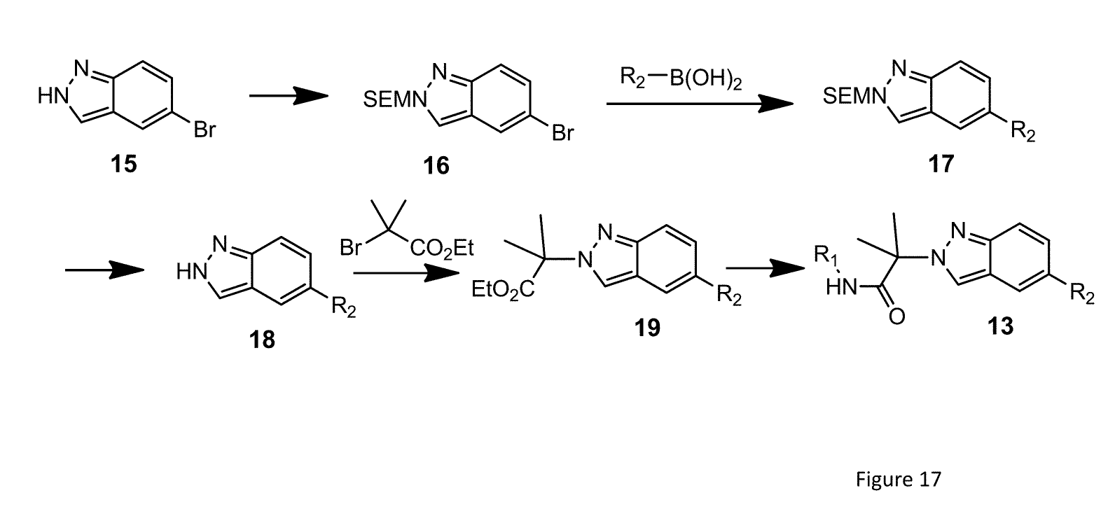

Compounds 9-11 are readily available through simple variations of the synthetic schemes shown in Figures b & c. Compounds 12 can be prepared from commercially available amino indoles using standard alkylation, acylation and protecting group chemistry. 2H-Indazoles (compounds 13) will be prepared using the scheme shown in FIG. 17.

Protection of commercially available compound 15 provides intermediate 16, which can be subjected to Suzuki coupling to give compound 17. Removal of the protecting group followed by a similar sequence to the one used for preparation of compounds 5 should give 2H-indazoles 13. A mixture of regioisomers might arise from the alkylation step, but literature precedence (Luo et al., 2006; Degnan et al., 2009) suggests that the 2-isomer of the indazole should predominate due to the hindered nature of the reaction and can be isolated by chromatography.

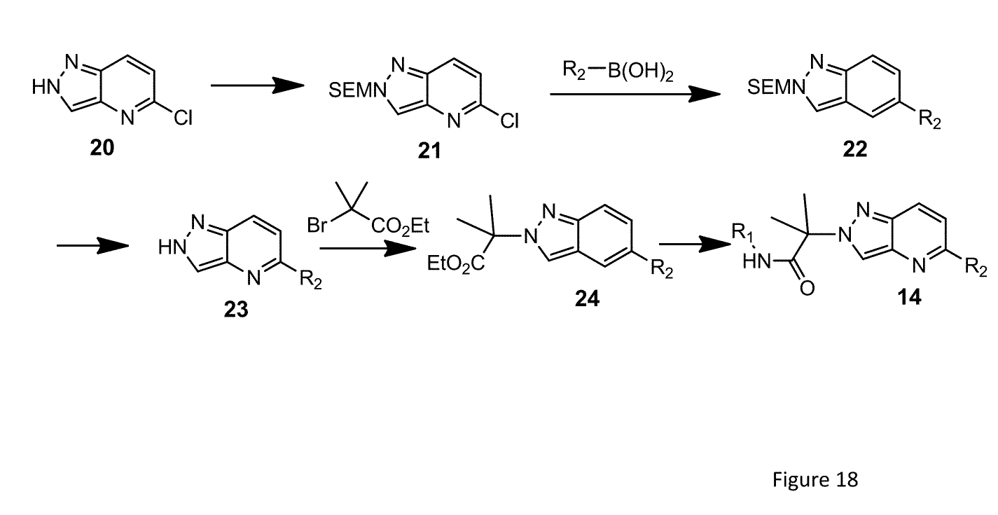

For preparation of pyrazolopyridines 14, treatment of intermediate 21 (prepared from commercially available compound 20) with boronic acids or esters under Suzuki conditions should provide intermediates 22, which can then be converted to pyrrazolopyridines 14 using a procedure similar to that described for the preparation of compounds 5 and 13 (FIG. 18). Again, the hindered nature of the alkylation reaction should favor formation of the 2-regioisomer, but any regioisomeric mixtures will be separated by chromatography.

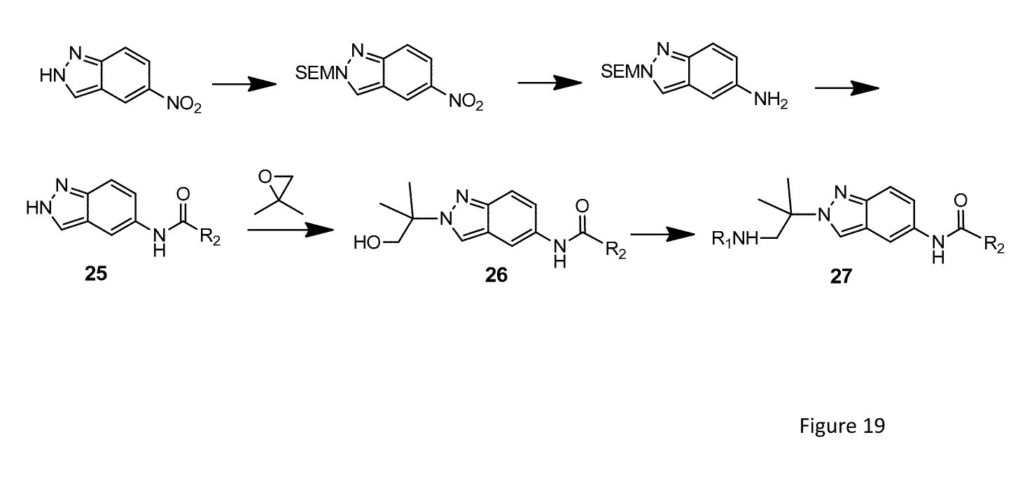

Analysis of the docking poses of SBC-110,424 suggest that the N—H groups of the two amides make hydrogen bonding interactions with residues on the protein, while only one of the carbonyl groups (flanking the 3-pyridyl) appears to engage a hydrogen bond. The corresponding amine analogs 27 were prepared in an attempt to enhance affinity by strengthening this hydrogen bond. The required amines can be prepared through the synthetic sequence shown in FIG. 19. Treatment of compounds 25 (available from commercially available 5-nitro-2H-indazole) with dimethyloxirane may provide intermediates 26, which can be converted to amines 27.

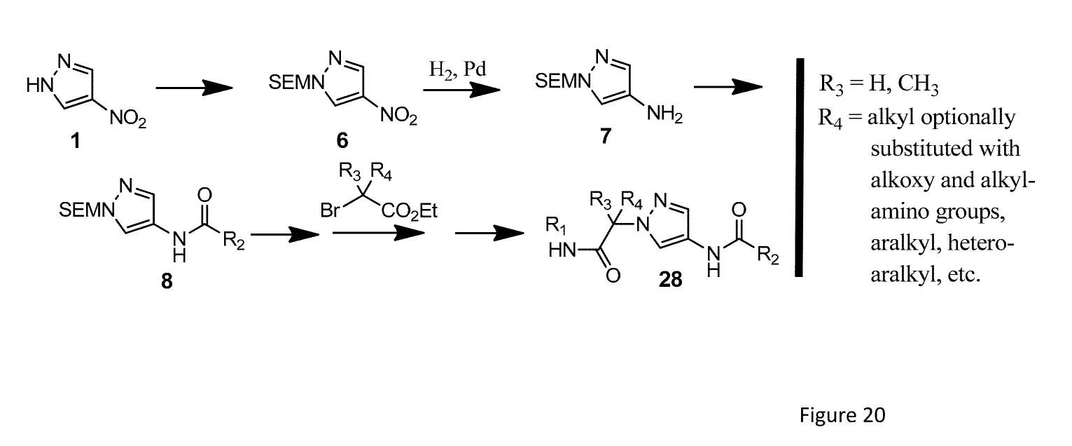

The germinal di-methyl groups adjacent to N1 of the pyrazole are involved in some non-bonded interactions of SBC-110,424 to PCSK9. One of the two methyl groups (the pro-(S) methyl) rests in a shallow lipophilic pocket. The pro-(R) position offers access to a cleft and other pockets on the protein that might provide additional binding interactions (FIG. 13, panel B). This structure may be used to increase the potency of the pyrazole series by performing SAR studies in this region. Various secondary and tertiary halides can be prepared and incorporated into the SBC-110,424 scaffold using a chemistry scheme shown in FIG. 20 to give compounds 28. Based on the regioselectivity seen in the reaction with ethyl-2-bromoisobutyrate (i.e., formation of intermediate 2), the 2-regioisomer can be expected to dominate in this reaction. The precedence set by this reaction also suggests that allylic and propargyl halides should successfully undergo the required substitution reaction. Due to the steric hindrance around the binding site of the pro-(S) substituent, substitution for R3 will be restricted to hydrogen and methyl. Choices for the pro-(R) substituent R4 can be driven by docking studies. Promising racemates can be separated into their enantiomers by chiral HPLC once bioactivity has been confirmed.

4-Hydroxy-5-oxopyrrole Analogs Lead Optimization

Docking studies suggest that the binding site of SBC-110,076 partially overlaps with that of SBC-110,424 (FIG. 13). The pyridine group appears to point toward solvent and makes no significant interactions. It would normally be prudent to remove this group to reduce molecular weight. However, there is no synthetic precedence for compounds with hydrogen or small alkyl groups in this position and the basic pyridine nitrogen atom provides a place for salt formation, so this group is left intact. The exocyclic carbonyl moiety is engaged in a hydrogen bond with the protein. The morpholine group seems to be forming a hydrogen bond through the oxygen atom as well as making van der Walls contacts with a neighboring disulfide bond in the protein. The tolyl group proximal to the exocyclic carbonyl lies in the same plane as one of the amides of SBC-110,424 and is nearly superpositioned with that same amide carbonyl, while the oxygen atom of the benzyloxy moiety occupies the binding site of the pyrazole nitrogens of SBC-110,424. The benzyloxy oxygen atom appears to be making hydrogen bonding interactions while the connected benzyl moiety makes lipophilic interactions with several protein residues. The phenyl moiety of the benzyloxy group lies at the entrance to the cleft bordered by the germinal dimethyl functionality of SBC-110,424. The initial strategy for optimizing the SBC-110,076 scaffold is to: 1) enhance the hydrogen bonding interaction of the morpholine oxygen atom by replacing it with substituted nitrogen-containing groups (i.e., piperazines); 2) install substituents and/or heteroatoms in the tolyl ring proximal to the exocyclic carbonyl to take advantage of the additional hydrogen bonds seen in the binding of SBC-110,424 (e.g., pyridyl); and 3) exploit the meta positions of the benzyloxy aryl group to access the aforementioned unoccupied pockets on PCSK9.

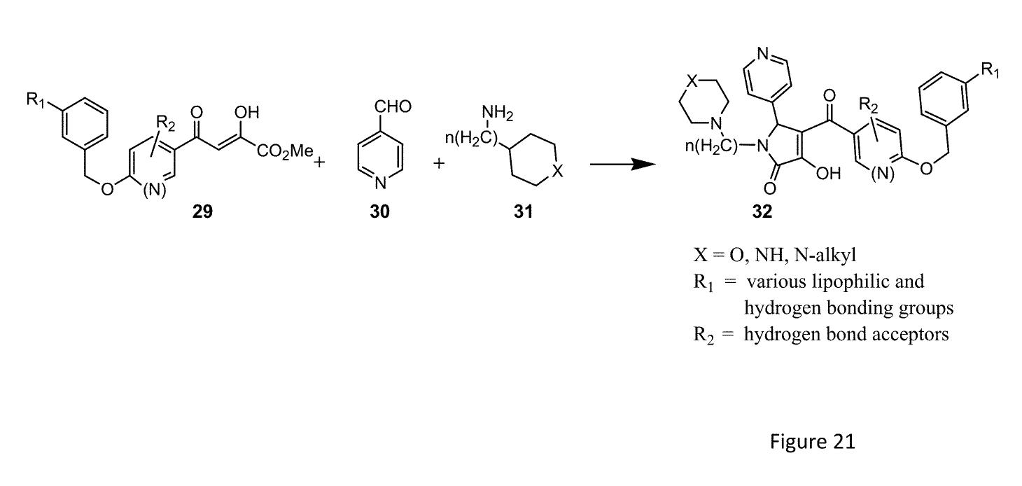

SBC-110,076 is chiral. Prior to beginning analog synthesis, SBC-110,076 can be separated into its respective enantiomers and identify the eutomer. For SAR studies, racemates can be tested initially and those that display good bioactivity can be subsequently separated into their respective enantiomers via chiral HPLC and retested. The general synthetic sequence shown in FIG. 21 can be used to synthesize the required analogs. The reaction can be carried out in one pot. One goal of the medicinal chemistry is to examine the SAR of the morpholine oxygen. This diversity can be accomplished by varying X in intermediate 31.

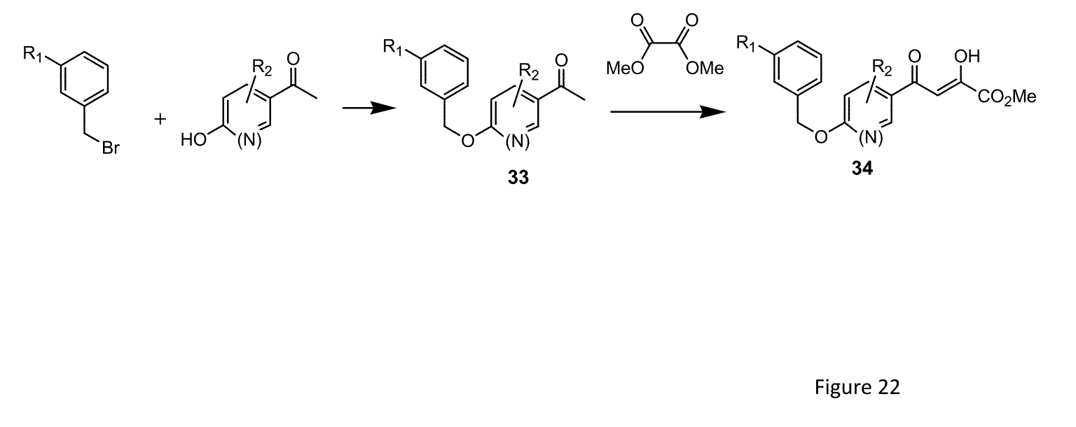

A second goal of the medicinal chemistry strategy is to build additional sites for hydrogen bond formation into the tolyl ring proximal to the exocyclic carbonyl group. This can be accomplished with heteroaryl groups such as pyridine, pyrimidine and pyrazine or through the use of hydrogen bonding accepting substituents on the aryl group such as halogen, trifluoromethyl and cyano (i.e., R₂). The third goal of the medicinal chemistry strategy is to establish additional interactions with the protein by accessing unoccupied clefts. This can be accomplished by building substituents (R₁) onto the meta position of the benzyloxy phenyl group. The diversity for goals 2 and 3 can be accomplished by synthesizing appropriate keto esters 34 using the sequence shown in FIG. 22.

Pending the outcome of each medicinal chemistry approach, the morpholino alkyl substituent can be removed, for example, if sufficient potency is achieved by one or more of the other approaches and more favorable physicochemical and ADME properties are needed.

The following assays can be used:

Plasma Stability—Compounds are assessed for their stability in mouse plasma at 37° C. Analysis is performed by LC/MS/MS on an API 4000 or Waters Xevo TQ instrument. MSMS analyses use positive or negative electrospray or APCI ionization using an API-4000. Assay acceptance criteria will be 20% for all standards and 25% for the LLOQ.

Aqueous solubility at pH 7.4—Compounds are assessed for their solubility at pH 7.4 using the commercially available Millipore MultiScreen™ Solubility filter system (Millipore, Billerica, Mass.). Analysis is performed using UV/VIS technology or LC/MS/MS as previously described.

Microsomal stability—Compounds are assessed for their microsomal stability by incubating them at 37° C. in the presence of liver microsomes and an NADPH regenerating system according to standard procedures. (Yang et al., 2005) Microsomal protein content is adjusted to give accurate rates of substrate consumption. Analysis is performed by LC/MS/MS as previously described. Assays are available using commercially available microsomes from several species, including human, mouse, rat, guinea pig, dot, monkey, minipig and rabbit. In addition, specialty pools of microsomes area available commercially through companies like Invitrogen (Carlsbad, Calif.).

Plasma protein binding—Plasma protein binding are measured by equilibrium dialysis under a CO2 atmosphere (Kochansky et al., 2008) and analyzed by LC/MS/MS as previously described.

Permeability assays—Permeability is assessed using an MDCK assay. The MDR1-MDCK uses standard procedures (Wang et al., 2005; Pastan et al., 1988), commercially available cells (Absorption Biosystems, Exton, Pa.) and 1 uM substrate concentrations to minimize transporter saturation. The MDCK-MR1 cell line is also used to monitor p-gp efflux liability. Results are analyzed by LC/MS/MS as previously described.

Any compounds that advance to in vivo efficacy studies can be examined in vivo for their pharmacokinetic parameters, including Cmax, AUC, half-life and bioavailability. These data can drive dose selection and time course and help establish a pharmacokinetic/pharmacodynamic relationship.

The disclosures of each and every patent, patent application, and publication cited herein are hereby incorporated herein by reference in their entirety. While this invention has been disclosed with reference to specific embodiments, it is apparent that other embodiments and variations of this invention may be devised by others skilled in the art without departing from the true spirit and scope of the invention. The appended claims are intended to be construed to include all such embodiments and equivalent variations.

1. A composition comprising a compound of Formula I: 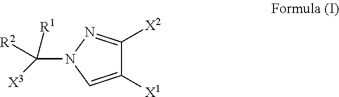
wherein in Formula (I):
R¹ is selected from the group consisting of hydrogen, C₁₋₆ alkyl, C₁₋₆ alkenyl, C₁₋₆ alkynyl, C₁₋₆ haloalkyl, optionally substituted aryl, and optionally substituted alkylaryl;
R² is selected from the group consisting of hydrogen, C₁₋₆ alkyl, C₁₋₆ alkenyl, C₁₋₆ alkynyl, C₁₋₆ haloalkyl, optionally substituted aryl, and optionally substituted alkylaryl, or R¹ and R² are optionally taken together with the atoms to which they are bound to form a ring containing 3 to 7 atoms, optionally containing a nitrogen, oxygen, or sulfur atom;
X¹ is selected from the group consisting of NR³COR⁴ and NR³SO₂R⁴
X² is selected from the group consisting of hydrogen and C₁₋₆ alkyl, or X¹ and X² are optionally taken together to form an optionally substituted aromatic six membered ring optionally containing a nitrogen atom;
X³ is selected from the group consisting of CONR³R⁴ and SO₂NR³R⁴;
R³ at each occurrence is independently selected from the group consisting of hydrogen and C₁₋₆ alkyl;
R⁴ at each occurrence is independently selected from the group consisting of hydrogen, C₁₋₆ alkyl, C₁₋₆ alkenyl, C₁₋₆ alkynyl, optionally substituted aryl, optionally substituted arylalkyl, optionally substituted heteroaryl, and optionally substituted heteroarylalkyl, or R³ and R⁴ are optionally taken together with the atoms to which they are bound to form a ring containing 3 to 7 atoms.

2. The composition of claim 1, wherein in Formula (I), R¹ and R² are methyl groups.

3. The composition of claim 1, wherein in Formula (I),
X¹ is NHCOR⁴; and
R⁴ is selected from the group consisting of optionally substituted aryl, optionally substituted arylalkyl, optionally substituted heteroaryl, and optionally substituted heteroarylalkyl.

4. The composition of claim 1, wherein in Formula (I),
X³ is CONHCH₂R⁵; and
R⁵ is selected from the group consisting of optionally substituted aryl, optionally substituted arylalkyl, optionally substituted heteroaryl, and optionally substituted heteroarylalkyl.

5. The composition of claim 1, wherein the compound of Formula (I) is a compound of Formula (II) or Formula (III):

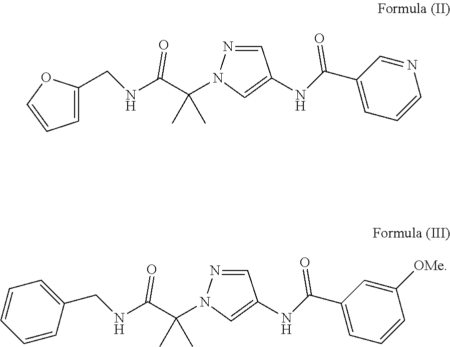 [SMILES: CC(C)(C(=O)NCc1ccco1)n1cc(NC(=O)c2cccnc2)cn1 ; COc1cccc(C(=O)Nc2cnn(C(C)(C)C(=O)NCc3ccccc3)c2)c1]

6. A composition comprising a compound of Formula (IV): 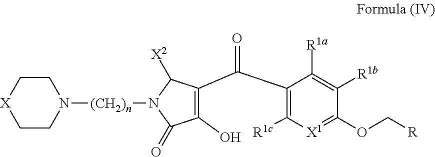
wherein in Formula (IV),
n is an integer from 1 to 6;
R is selected from the group consisting of optionally substituted aryl and optionally substituted heteroaryl;
R^{1a}, R^{1b}, R^{1c}, and R^{1d} are at each occurrence independently selected from the group consisting of hydrogen, halogen, optionally substituted C₁₋₆ alkyl, optionally substituted C₁₋₆ alkenyl, optionally substituted C₁₋₆ alkynyl, optionally substituted C₁₋₆ haloalkyl, optionally substituted C₃₋₇ cycloalkyl, optionally substituted C₁₋₆ alkoxy, cyano, nitro, OR², SR³, SO₂R³, NR^{4a}R^{4b}, NR^{4a}COR⁵, NR^{4a}CONR^{4a}R^{4b}, NR^{4a}COOR⁶, SO₂NR^{4a}R^{4b}, and NR^{4a}SO₂R⁶;
X is selected from the group consisting of oxygen, sulfur, NR^{4a}, NCOR⁵, NCONR^{4a}R^{4b}, NCOOR⁶, and NR^{4a}SO₂R³;
X¹ is selected from the group consisting of N and CR^{1d};
X² is selected from the group consisting of optionally substituted aryl and optionally substituted heteroaryl;
R² at each occurrence is independently selected from the group consisting of hydrogen, optionally substituted C₁₋₆ alkyl, optionally substituted C₁₋₆ alkenyl, optionally substituted C₁₋₆ alkynyl, optionally substituted C₁₋₆ haloalkyl, optionally substituted C₃₋₇ cycloalkyl, optionally substituted aryl, COR⁵, CONR^{4a}R^{4b}, SO₂NH₂, and SO₂R³;
R³ at each occurrence is independently selected from the group consisting of hydrogen, optionally substituted C₁₋₆ alkyl, optionally substituted C₁₋₆ alkenyl, optionally substituted C₁₋₆ alkynyl, optionally substituted C₁₋₆ haloalkyl, optionally substituted C₃₋₇ cycloalkyl, and optionally substituted aryl;
R^{4a} and R^{4b} at each occurrence are independently selected from the group consisting of hydrogen, optionally substituted C₁₋₆ alkyl, optionally substituted C₁₋₆ alkenyl, optionally substituted C₁₋₆ alkynyl, optionally substituted C₁₋₆ haloalkyl, optionally substituted C₃₋₇ cycloalkyl, and optionally substituted aryl;
R⁵ at each occurrence is independently selected from the group consisting of hydrogen, optionally substituted C₁₋₆ alkyl, optionally substituted C₁₋₆ alkenyl, optionally substituted C₁₋₆ alkynyl, optionally substituted C₁₋₆ haloalkyl, optionally substituted C₃₋₇ cycloalkyl, and optionally substituted aryl; and
R⁶ at each occurrence is independently selected from the group consisting of optionally substituted C₁₋₆ alkyl, optionally substituted C₁₋₆ alkenyl, optionally substituted C₁₋₆ alkynyl, optionally substituted C₁₋₆ haloalkyl, optionally substituted C₃₋₇ cycloalkyl, and optionally substituted aryl.

7. The composition of claim 6, wherein in Formula (IV), n is 3 and X is oxygen.

8. The composition of claim 6, wherein in Formula (IV), X² is 4-pyridyl.

9. The composition of claim 6, wherein in Formula (IV), R is phenyl.

10. The composition of claim 6, wherein the compound of Formula (IV), is a compound of Formula (V):

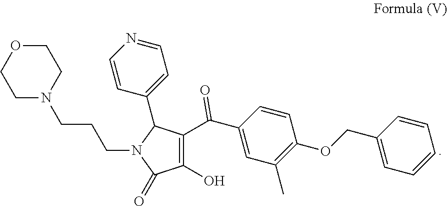 [SMILES: Cc1cc(C(=O)C2=C(O)C(=O)N(CCCN3CCOCC3)C2c2ccncc2)ccc1OCc1ccccc1]

11. A method for inhibiting proprotein convertase subtilisin-like kexin type 9 (PCSK9) in a subject, comprising:
administering a therapeutically effective amount of the composition of claim 1 to a subject.

12. A method for disrupting the protein-protein interaction between PCSK9 and low-density lipoprotein receptor (LDLR), comprising:
administering a therapeutically effective amount of the composition of claim 1 to a subject.

13. A method for treating a lipid disorder in a subject, comprising:
administering a therapeutically effective amount of the composition of claim 1 to a subject.

14. The method of claim 13, wherein the lipid disorder is hypercholesterolemia.

15. The method of claim 13, wherein the composition further comprises an additional therapeutic agent selected from the group consisting of an HMGCoA reductase inhibitor, a nicotinic acid, a fibric acid, and a bile acid-binding resin.

16. A method for inhibiting proprotein convertase subtilisin-like kexin type 9 (PCSK9) in a subject, comprising:
administering a therapeutically effective amount of the composition of claim 6 to a subject.

17. A method for disrupting the protein-protein interaction between PCSK9 and low-density lipoprotein receptor (LDLR) in a subject, comprising:
administering a therapeutically effective amount of the composition of claim 6 to a subject.

18. A method for treating a lipid disorder in a subject, comprising:
administering a therapeutically effective amount of the composition of claim 6 to a subject.

19. The method of claim 18, wherein the lipid disorder is hypercholesterolemia.

20. The method of claim 18, wherein the composition further comprises an additional therapeutic agent selected from the group consisting of an HMGCoA reductase inhibitor, a nicotinic acid, a fibric acid, and a bile acid-binding resin.
